(19) United States
(12) Patent Application Publication
(10) Pub. No.: US 2016/0239852 A1
(43) Pub. Date: Aug. 18, 2016

(54) MULTICOMMODITY SYSTEM AND METHOD FOR CALCULATING MARKET DYNAMICS IN HEALTH NETWORKS SYSTEMS

(71) Applicant: PokitDok, Inc., San Mateo, CA (US)

(72) Inventors: Theodore Tanner, JR., San Mateo, CA (US); Denise Koessler Gosnell, San Mateo, CA (US)

(73) Assignee: PokitDok, Inc.

(21) Appl. No.: 14/941,045

(22) Filed: Nov. 13, 2015

Related U.S. Application Data
(63) Continuation of application No. 14/625,482, filed on Feb. 18, 2015.

(51) Int. Cl. G06Q 30/02; G06F 17/30; G06Q 10/08

(52) U.S. Cl. CPC G06Q 30/0201; G06Q 10/087; G06F 17/3053; G06F 17/30604

(57) ABSTRACT

A multi-commodity system and method for calculating market dynamics in health network systems are disclosed. The system and method may incorporate a process that computes the influence of several transactional based entities within a business based health architecture. The system and method may: (1) calculate the diffusion of information theoretic data, (2) compute the influencers in each of the area of business driven health sectors and (3) optimize entity matching likelihoods across health network silos.

### PRIORITY CLAIM

This application is a continuation of and claims priority under 35 USC 120 to U.S. patent application Ser. No. 14/625,482, filed on Feb. 18, 2015 and entitled “Multi Commodity System and Method for Calculating Market Dynamics in Health Network System”, the entirety of which is incorporated herein by reference.

### APPENDICES

Appendix A (6 pages) is an example of the code that calculates similarity scores and rank entities for a homogeneous system.

Appendix B (9 pages) contains several examples of raw data showcasing service data.

Appendices A and B form part of the specification and are incorporated herein by reference.

### FIELD

The disclosure relates generally to a system and method for calculating market dynamics and in particular to a system and method for calculating market dynamics in health network systems.

### BACKGROUND

In most cases, for a traditional multicommodity flow problem, there are multiple commodities across a single network so the total amount of flow on each edge is no more than the maximum capacity of the edge. However, these traditional multicommodity flow solutions do not work well for multiple networks with multiple commodity flows calculating supply and demand with multiple network and sub-network flows. For these more complex problems, it is possible to express the problem as a linear programming model. However, most models of this nature fall short with respect to large scale modeling and the large size of the linear programs makes the general simplex algorithm impractical for all but very small problems.

An example of a typical health network in shown in FIG. 1. The health network may include a provider or physician network and the influencers or directors of medicine were found to have significantly denser, more cohesive and more horizontal social networks and to be members of significantly more professional organizations. This may cause a very slow adoption and clique based influencing and recommendation. This, in large part, is due to the fact that the main influencers are the opinion leaders cannot manage or endorse technology or information whereas their social network is very egalitarian and see their decision making as highly autonomous.

To exacerbate the problem within a business health network, the technology that drives claims and benefits health care processing in the industry is known as ASC 5010 X12. This is an Electronic Data Interchange standard for the industry. The ASC 5010 X12 standard, however, creates silos within the health network topologies.

The problem of measuring supply and demand within various capital markets are well known and systems and methods exist that address this capital markets problem. However, there are not any systems for calculating commodity flow influence for transactional business models within Health Related entities such as a provider, a consumer, a service and a transaction. Furthermore, the known capital market techniques cannot be used for health related entities because the health related entities are unique and have problems that did not exist until health services marketplaces took hold.

FIG. 2 illustrates an example of a single commodity flow system. In the case of modeling of the Single Commodity Flow (SCF) system, the known modeling is based on a directed flow network or a Directed Graph where the graph is defined as:

G=(V,E)

in which E is an edge and V is a vertex and
each edge E has a capacity cₑ
a source node s ∈ V
a sink node t ∈ V

A flow f is a directed graph as stated that has the same vertices of G, where every single edge has a value spanning from 0 to cₑ where cₑ is the capacity of that specific edge. Formally, a solution to the Single Commodity Flow System is a mapping f: E→R⁺, denoted f(u,v) or fᵤᵥ which assigns to every edge (u,v) Å E a non-negative flow value x_{u,v}≧0 such that the following two fundamental flow constraints are conserved:
a. Capacity Constraint: f(u,v)≧c(u,v) ∀(u,v) ∈ E
b. Conservation of Flow: Σ_{u,v∈E} f(u,v)=Σ_{v,u∈E} f(v,u)

The structure of the Single Commodity Flow System describes the simplest view of modeling the diffusion of entities throughout a network.

In a historical case, suppose that a company has a factory s and a warehouse t. The quantity of goods that the company can ship from the factory to the warehouse in a given time period is limited by the road network connecting the two facilities. The company wants to determine the maximum quantity of goods that can be shipped through the road network. This scenario is a case of the standard max-flow problem for a single commodity network in which the single commodity is quantity of goods. It is well known that the max-flow is equal to the min-cut (described in Ahlswede, Rudolf, et al. “Network information flow.” Information Theory, IEEE Transactions on 46.4 (2000): 1204 -1216 which is incorporated herein by reference), which, in this case, would be the set of roads with the smallest capacity such that removing the roads disconnects the graph. In this theoretic example, perhaps it is a collection of bridges that cause a bottleneck when trying to cross the local river. However, the SCF system is inadequate for the complex health network systems with multiple commodities as described above.

FIG. 3 illustrates an example of a multiple commodity flow system. For modeling the Multicommodity Flow Network (MCF) shown in FIG. 3, the input to consists of n nodes, m edges and k commodities, each with a source sink and a demand of the corresponding O(nk) variables and O(nk+m) constraints. Formally, a multicommodity flow network may have a graph defined as G=(V,E) with
pairs of vertices sᵢ,tᵢ ∈ V, each representing a source and a sink for commodity i
A demand Dᵢ for each commodity
A capacity function C on the edges of G such that

$${\sum }_{all i} {f}_{i} ( e ) \leq {c}_{e}$$

A solution to the Multicommodity Flow System assigns, to every edge e ∈ E for every commodity k ∈ K, a non-negative flow value x_{e,k}≧0 such that the following principles of flow are obeyed:
c. Capacity Constraint: f(u,v)≧c(u,v) ∀(u,v) ∈ E
d. Conservation of Flow:Σ_{u,v∈E} f(u,v)=Σ_{v,u∈E} f(v,u)

The design of the Multicommodity Flow System enables us to model the diffusion of multiple entities throughout a network with a single entrance and exit. However, this static MCF system described above is also inadequate. In particular, the above static MCF cannot model the inherent dynamic nature of the healthcare system along with ever changing source and sink targets.

The multicommodity flow system may be used for a company that has multiple factories that each make a different product to distribute to several warehouses, each warehouse has a fluctuating demand for each of the products and each product has a different distribution frequency. It is desirable to determine the same solution as before; however, the system is constrained to model a maximum distribution from within a multivariate and dynamic network.

As such, the concurrent maximum flow can be defined to be the largest fraction f such that the method can fill a fraction fᵢ of each of these demands simultaneously and dynamically. However, the static MCF system cannot handle the diffusion of healthcare information that perpetrates throughout the network in a dynamic manner in both type and source.

A known Dynamic Multicommodity Flow System is a directed network N=(V,E,K,T,ω,μ,T,d,φ) with set of vertices V, directed edges E and set of commodities K. Further, each edge e ∈ E has a nonnegative time-varying capacity ω_{k,e}(t) which bounds the total flow for each commodity kover each edge e ∈ E during time t ∈ T. Over all commodities k ∈ K, each edge is further constrained by the mutual edge capacity μₖ^{∈}(t) such that Σᵢ₌₀^{k} ω_{i,∈}(t)≧μ_{k∈}(t) Additionally, each edge has a transmit time τ₅₃₁ which determines the time it takes for the commodities to flow from the source to the destination of the corresponding edge. Lastly, the entire network also consists of a demand function dᵢV×K×Γ→R₊ and a cost function φ; E×R₊×K×Γ→R₊, where Γ={0,1, . . . T}

In a Dynamic Multicommodity Flow System, the demand function d_{v,k}(t) is constrained to the following conditions:
a. there exists a v ∈ V for every k ∈ K such that d_{v,k}(0)<0;
b. if d_{v,k}(t)<0 for a node v ∈ V for commodity , then d_{v,k}(t)=0,t=1.2, . . . T.

In order to model flow in this network, the existence of a flow equilibrium is required. That is, Σ_{t ∈ Γ} Σ_{v ∈V} d_{v,k}(t)=0,∀k ∈ K. Further, the sources of this network are those vertices with negative demand, Σ_{t ∈ Γ} d_{v,k}(t)<0, the sinks of this network are those vertices with positive demand: Σ_{t ∈ Γ} d_{v,k}(t)>0 and the intermediate nodes are those with zero demand: Σ_{t ∈Γ} d_{v,k}(t)=0. The sources are the nodes through which flow enters the network and the sinks are the nodes through which flow exits the network. The intermediate nodes are transport nodes.

In a Dynamic Multicommodity Flow System, the cost function also takes into account the transit cost of a commodity k throughout the network with the function φ_{k,∈}(x_{k,∈}(t),t); meaning that the flow of commodity kof value x_{k,e}(t) entering edge e at time t will incur a transit cost of φ_{k,e}(x_{k,e}(t),t).

As such, the total cost of the dynamic multicommodity flow xis defined as:

c(x)=Σₜ₌₀^{T} Σ_{k∈K} Σ_{e ∈E} φ_{k,e}(x_{k,∈}(t),t) [Equation 1]

The Multicommodity Dynamic Flow problem is to find a feasible flow that minimizes the objective function as shown in Equation 1.

### BRIEF DESCRIPTION OF THE DRAWINGS

FIG. 1 illustrates an example of a typical healthcare network;

FIG. 2 illustrates an example of a single commodity flow system;

FIG. 3 illustrates an example of a multiple commodity flow system;

FIG. 4 illustrates an example of a flow for multiple commodities;

FIG. 5A illustrates an example of a health network system that may incorporate a market dynamics component;

FIG. 5B illustrates more details of a market dynamic component;

FIG. 6 illustrates more details of the market dynamic component and its update and feedback loops;

FIG. 7 illustrates an example of a set of homogeneous relationships in a health care network;

FIGS. 8 and 8A illustrate an example of a method for ranking an entity based on the health graph;

FIG. 9 illustrates an example of a set of heterogeneous relationships in a health care network;

FIG. 10 illustrates a method for computing multicommodity flow for a healthcare network;

FIGS. 11 and 12 illustrates examples of heterogeneous influence models;

FIG. 13 illustrates an example of a schema for the graph construction and storage centered on the consumer model;

FIG. 14 illustrates an example of a schema for the graph construction and storage centered on the provider model;

FIG. 15 illustrates an example of a schema for the graph construction and storage centered on the payor model;

FIG. 16 illustrates an example of a schema for the graph construction and storage centered on the service model;

FIG. 17 illustrates an example of a schema for the graph construction and storage centered on the transaction model;

FIG. 18 is an example of the code that may create the schema and load the schema into a graph;

FIG. 19 illustrates an example of the data interchange between two entities in the healthcare network;

FIG. 20 illustrates two uses cases of a commodity network in the healthcare field;

FIG. 21 illustrates an example of a dynamic multicommodity healthcare network from which the commodity flow may be calculated using the system shown in FIGS. 5A and 5B;

FIG. 22 illustrates an example of pseudocode for flow estimation used in the model;

FIGS. 23-25 illustrate the processes performed by the component shown in FIG. 5B; and

FIG. 26 illustrates a commodity flow example when Jane Doe receives service from John Doe with Mockpayor support.

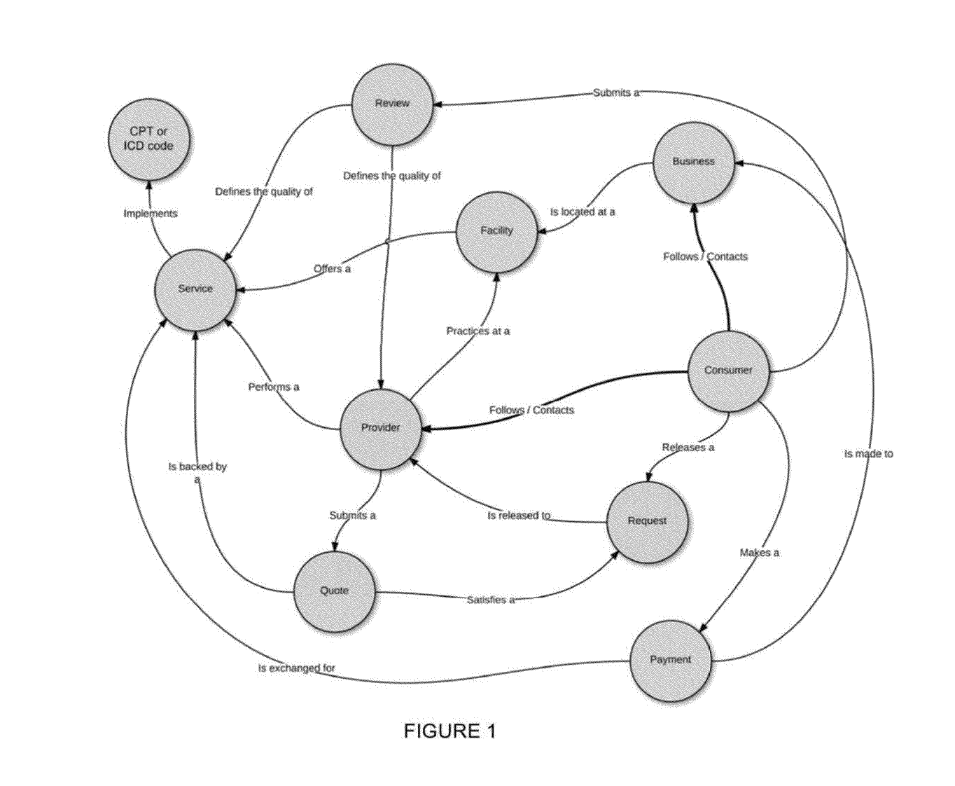

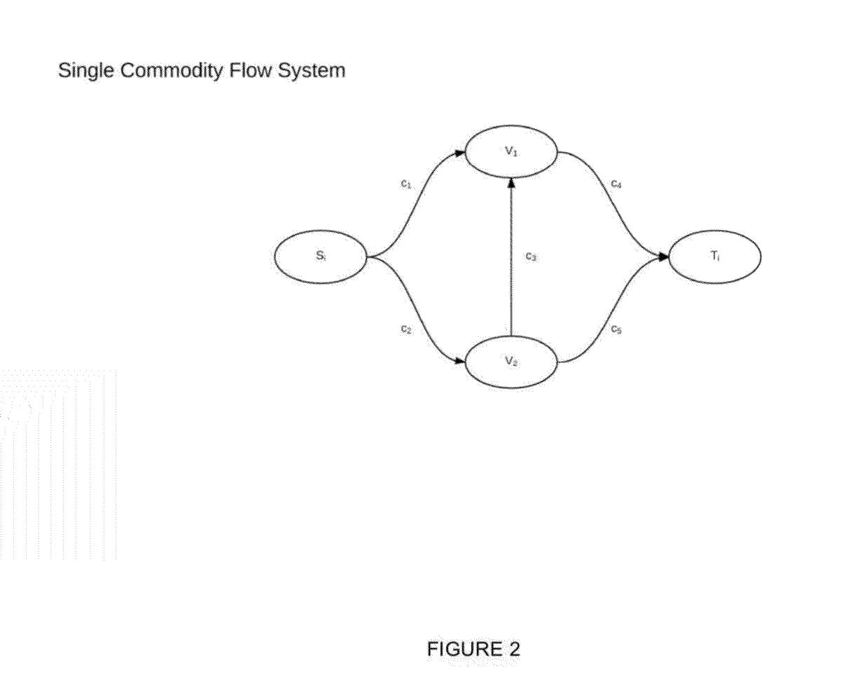

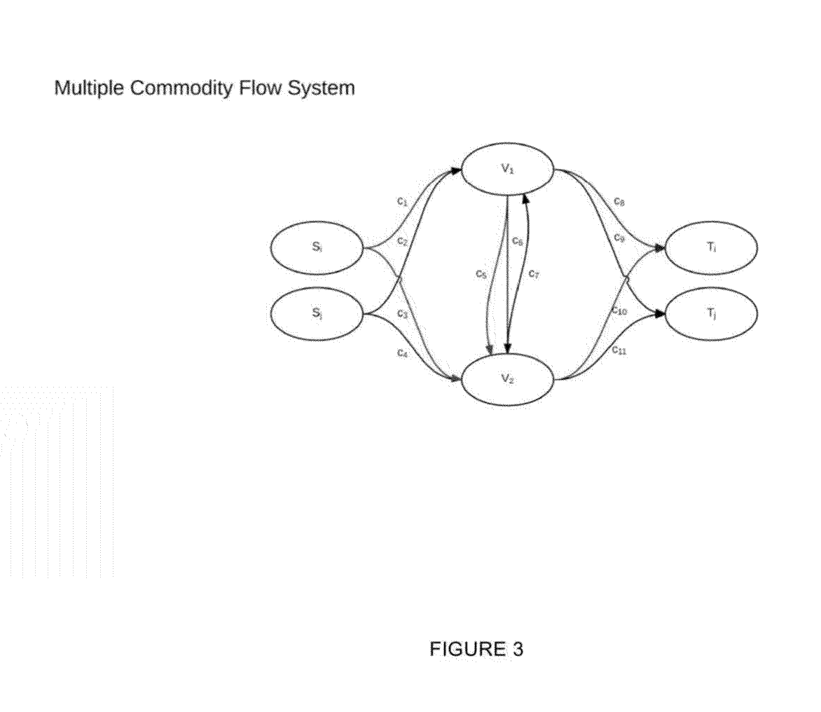

### DETAILED DESCRIPTION OF ONE OR MORE EMBODIMENTS

The disclosure is particularly applicable to a multi-commodity system for health networks implemented in a SaaS or client/server architecture and it is in this context that the disclosure will be described. It will be appreciated, however, that the system and method has greater utility since the system and method can be implemented in other manners that are also within the scope of the disclosure.

The system and method may be used to calculate the rate of change for the influence of supply and demand with respect to several multi-variate factors for the practitioner, payer and consumer alike in a health network. The system and method may have a process that computes the influence of several transactional based entities within a business based health architecture. The influence score may be calculated from within each homogeneous system and used to infer the likelihood of connectivity throughout the entire heterogeneous model.

There has been much literature covering diffusion of health innovations in areas such as pharmaceuticals, devices and medical services (Medical Innovation-A Diffusion Study, Coleman, J. S., Katz, E., Menzel, H., Bobbs-Merrill Company, 1966; and Diffusion of Innovations In Health Service Organizations-A Systematic Literature Review, Greenhalgh, T., Glenn, R., Bate, P., Macfarlane, F., Kyriakidou, O., Blackwell Publishing, 2005). The system and method disclosed below may calculate the diffusion of information theoretic data, compute the influencers in each of the areas of business driven health sectors and optimize entity matching likelihoods across health network silos. For example, the system and method may be used for a healthcare network for market index prediction, target market segmentation and a myriad of other market driven applications.

The system described below may be used in the health sector that has social and professional networks within the health sector. In one exemplary implementation, a search topology that encompasses 4.1 million health care providers was created. The providers include acupuncture, chiropractors, surgeons and general practitioners in all of the different health specialties. With respect to these providers, the professional communications through which information, influence and innovation occur is bounded. Further, the flow of information exchange and influence is deeply obfuscated within the networks and subnetworks. In the system, the these networks may be modeled with a graph topology. The graph topology coalesces claims and benefits transactions, payment exchanges, payers [insurers] and consumer behavior.

With the graph topology, the system and method uses the observed data exchanges to extract behaviors, derive individual influence and model the cascade of information exchange. The calculation and prediction of influence throughout the network may be performed by the system using techniques known as Multi Commodity Flow Algorithms.

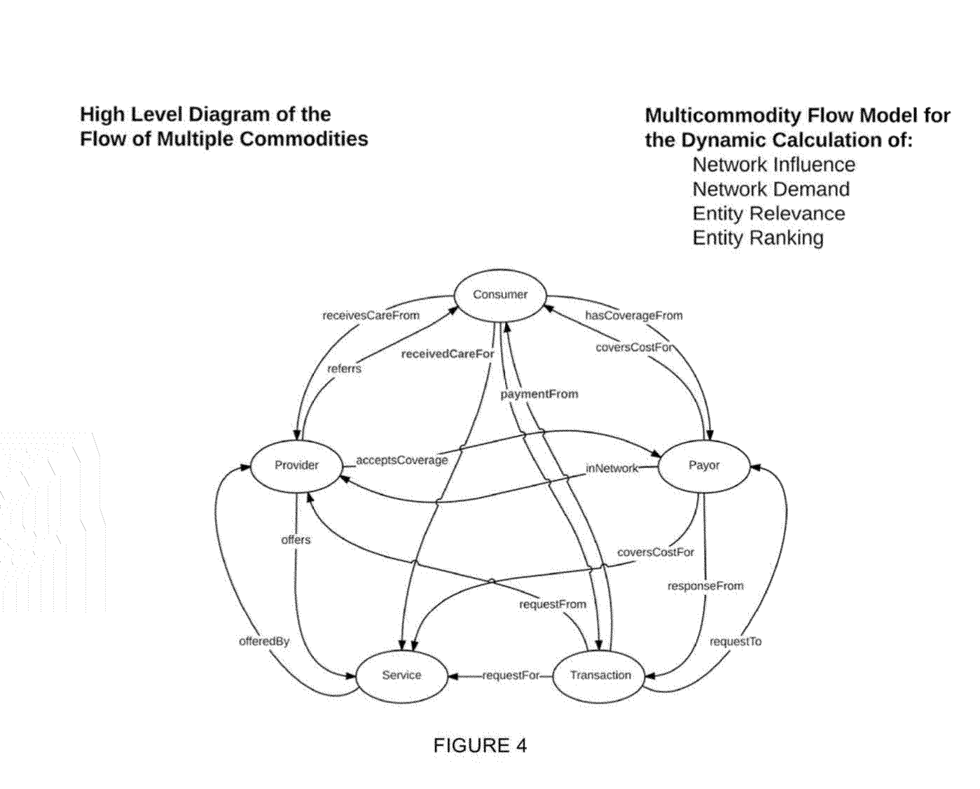

FIG. 4 illustrates an example of a flow for multiple commodities in a health sector. Thus, the flows may be used for supply and demand models for Health Services Networks (a “HealthGraph”). The system may model these commodity flow relationships with known diffusion or cascade algorithms. The system can calculate the dynamic distribution of multiple commodities based on the dynamic influence of individual or clustered networks. These diffusion network models may be derived from interactions within the health network from a payer, provider and consumer interaction mechanism as are shown in FIG. 4 in which entities (such as provider, consumer, payor, service and transaction) are shown by ovals and interactions (such as offeredBy, requestFor, coversCostFor, etc) are shown by the arrows that connect the ovals. A direction of the interaction between two entities are shown by the direction of the arrow (such as a requestFor interaction from the transaction entity to the service entity or a offeredBy interaction from the service entity to the provider entity).

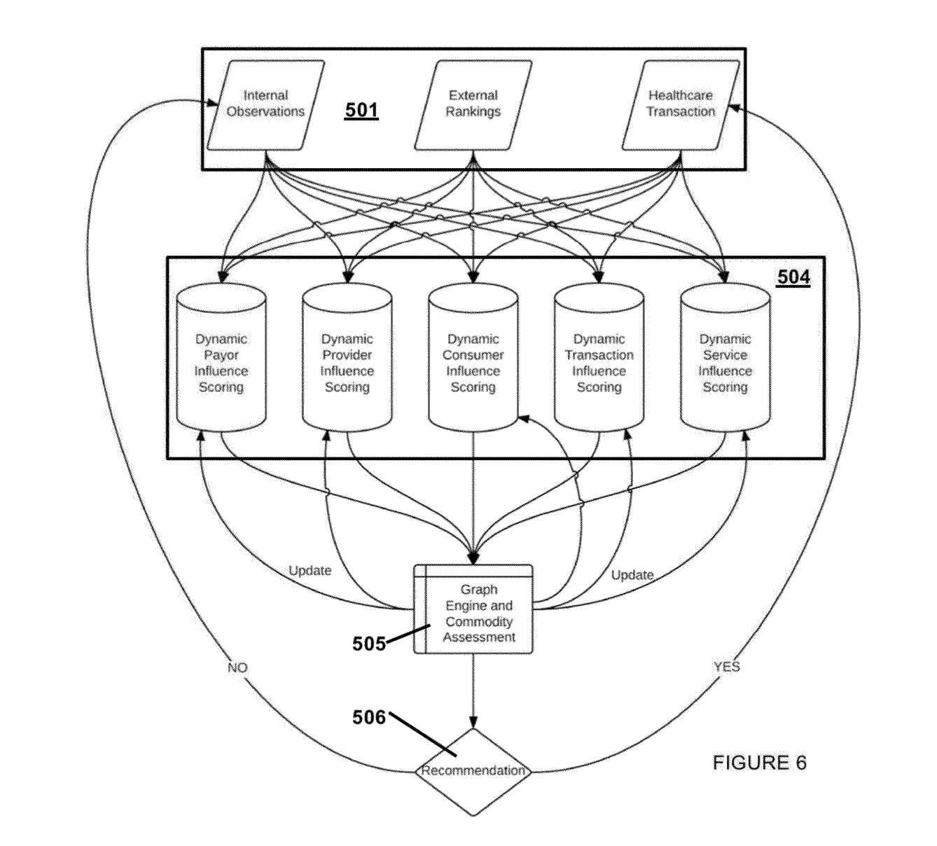

The system allows calculations of information cascades across several vertically oriented networks within the overall operational aspects being ensconced in a graph theoretic persistence and calculation. This information cascade calculation is specifically aimed at calculating the commodity flow within these otherwise siloed networks. As shown in FIG. 6, the siloed networks may be a payor network, a provider network, a consumer network, a transaction network and a service network in the health sector.

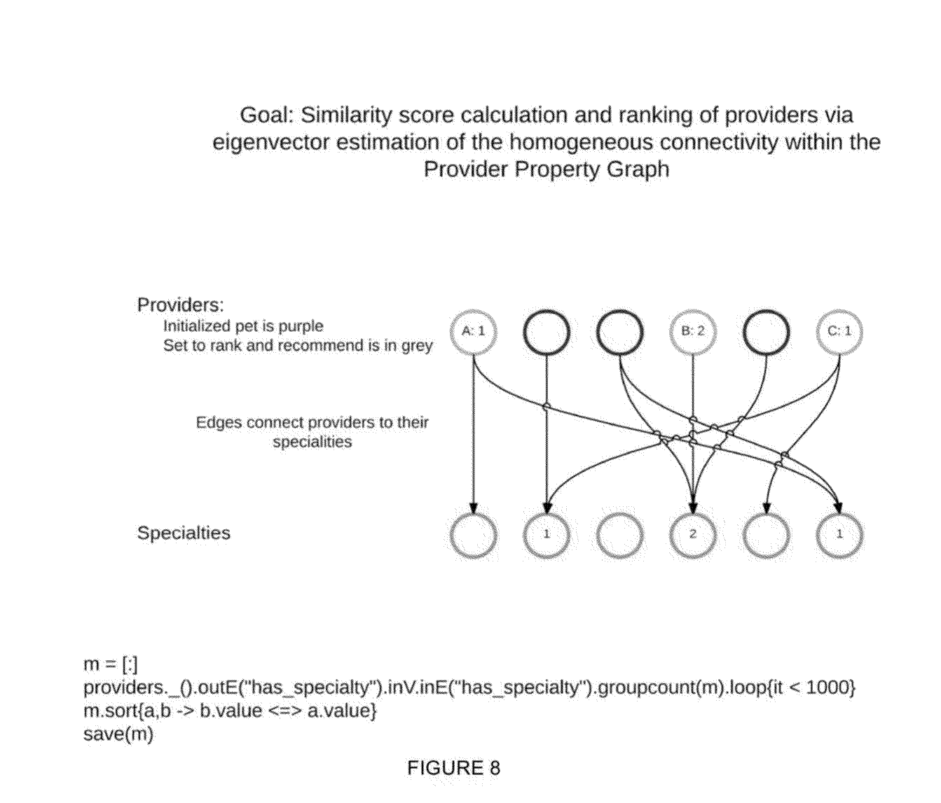

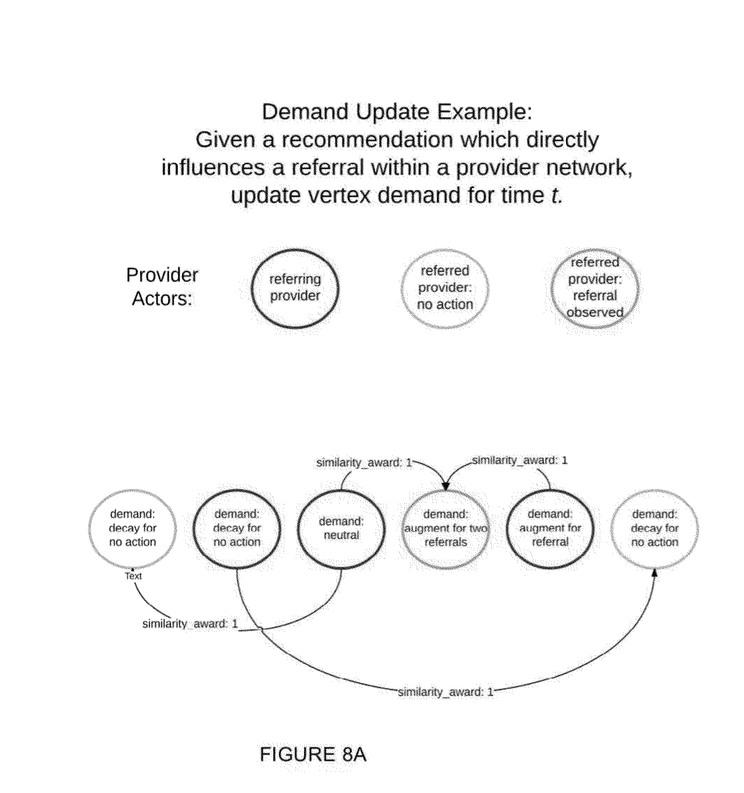

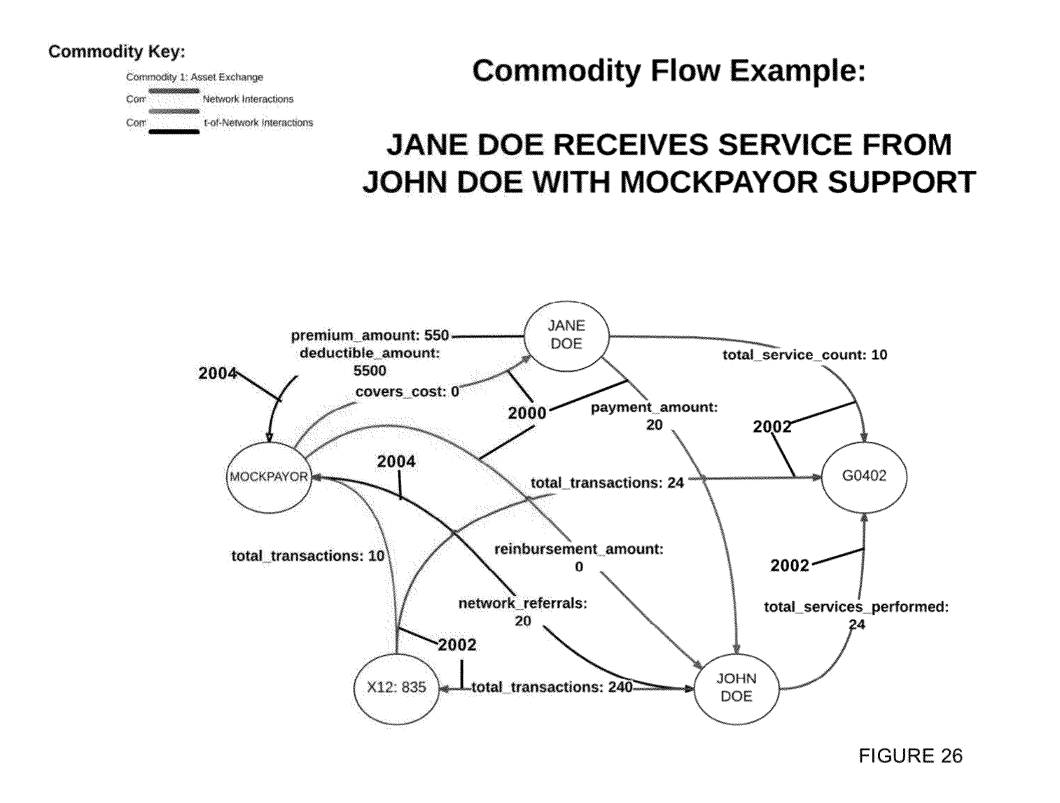

Returning to FIG. 4, the system and method may be used for the multiple commodity flow in FIG. 4 for:
Automatically setting the services pricing indexes for cash based as well as re-imbursed contracted service rates. For example, based on the average asset exchange observed in the network, as demonsrated in FIG. 26, we can release the average out of pocket price, average reimbursement, average co-pay and average cash price per service.
Automatically matching any of the homogenous networks in a cross heterogenous fashion whereas a provider is matched to a consumer or a consumer is matched to a payor. For example, the system can apply the ranking and referral techniques demonsrated in FIG. 8 and FIG. 8A to match similar consumer and provider pairs based on common services observed in transactions, as demonstrated in FIG. 26.
Targeted segmentation of demographic behaviors for marketing applications or content deployment. For example, the system can apply the ranking and referral techniques demonsrated in FIG. 8 and FIG. 8A to match similar consumer and provider pairs based on common services observed in transactions, as demonstrated in FIG. 26.

Based on FIG. 4, the system may also dynamically calculate network influence, network demand, entity relevance and/or entity ranking as described below in more detail.

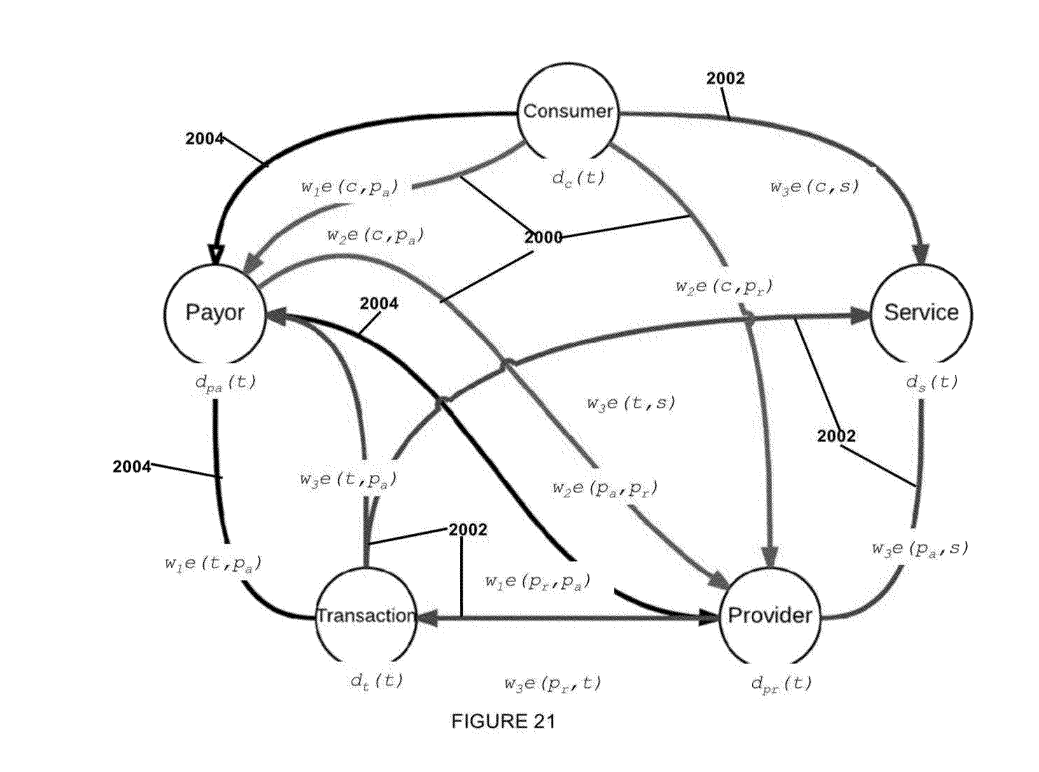

The system and method described below may be used to solve the multicommodity dynamic flow problem over a health network with non-disjoint and dynamic sets of source, sink and transport nodes (an example of which is shown in FIG. 21). In contrast to the traditional approach that categorizes each vertex according to one demand function Σ_{t ∈ Γ} d_{v,k}(t), the system and method provides a solution that allows dynamic and co-occurring classifications of a vertex's demand.

Given a dynamic multicommodity network, the system and method seeks to estimate the amount of flow capacity ω_{k,∈}(t) for each edge e for k commodities throughout time T while adhering to the demand and capacity constraints. The instance of the problem being solved by the system and method and its solution may be formally framed as follows:

Given a graph G=(V,E,K,T,ω,T, d) where each vertex v ∈ V has dynamic weight d_{k,v}(t) for commodity k ∈ K and each vertex is both a source and a sink for the flow of commodity k ∈ K in N(vᵢ), find an assignment of flow capacities ω_{k,∈}(t) which satisfies the following constraints:

1. Capacity constraints: Σᵢ₌₀^{k} ω_{i,∈}(t)≦μ_{∈}(t) , where μ_{k,∈}(t) is the universal capacity allowed on edge e at time t (the flow of a vertex cannot exceed its total capacity)

2. Flow conservation: rₖ(t)=Σ_{v∈N(v)} ω_{k,e}(t)=d_{k,N}(t) (the sum of the flow exiting a node and the remaining flow equal the original vertex capacity) If the measure of diffusion for vertex v ∈ V is represented by rₖ(t) and the value of flow is given by |f|=Σ_{v∈N(v)} ω_{k,e}(t) , then the maximal diffusion estimation for this instance of a Dynamic Multicommodity Network is to maximize |f| such that Σ_{v∈N(v)} rₖ(t)→0.

The system and method uses methodologies in social influence and entity ranking to model optimal matchings, predict market indices and create target segmentation within a healthcare network. The importance of social influence and entity ranking within the context of social and economic networks is well documented and understood (see Jackson, Matthew O. Social and economic networks. Vol, 3. Princeton: Princeton University Press, 2008: and Jackson, Matthew O., and Alison Watts. “The evolution of social and economic networks,”Journal of Economic Theory 106.2 (2002): 265-295, both of which are incorporated herein by reference). The system and method apply dynamic entity ranking through application of multicommodity flow within a health network.

In a healthcare network, the system and method dynamically models the flow of a myriad of commodities throughout various sets of entities (as shown in FIG. 4) to estimate and predict the demand of products. To do so, the system may partition V into disjoint sets of vertices:

V ⊂ {Pₐ,Pᵣ,C,S,T}, [Equation 2]

where the sets are defined as follows: Pα are the payors, Pᵣare the providers, C are the consumers, S are the services and T are the transactions observed within the healthcare network as shown in FIG. 4. The system may dynamically apply methodology in social network ranking algorithms (an example of which is disclosed in Wasserman, Stanley. Social network analysis: Methods and applications. Vol. 8. Cambridge university press, 1994 which is incorporated herein by reference) to calculate the total influence within each set of homogeneous entities in our the healthcare network. The influence score for a vertex is applied to calculate the flow of multiple commodities throughout the entire health network as shown in FIG. 21. The calculation of the dynamic influence of multiple commodities within a health network enables the system to model and predict the ebb and rise of: service reimbursement, service cost, service supply and demand, payment trends, market indices, optimally matched bi-partite systems and more.

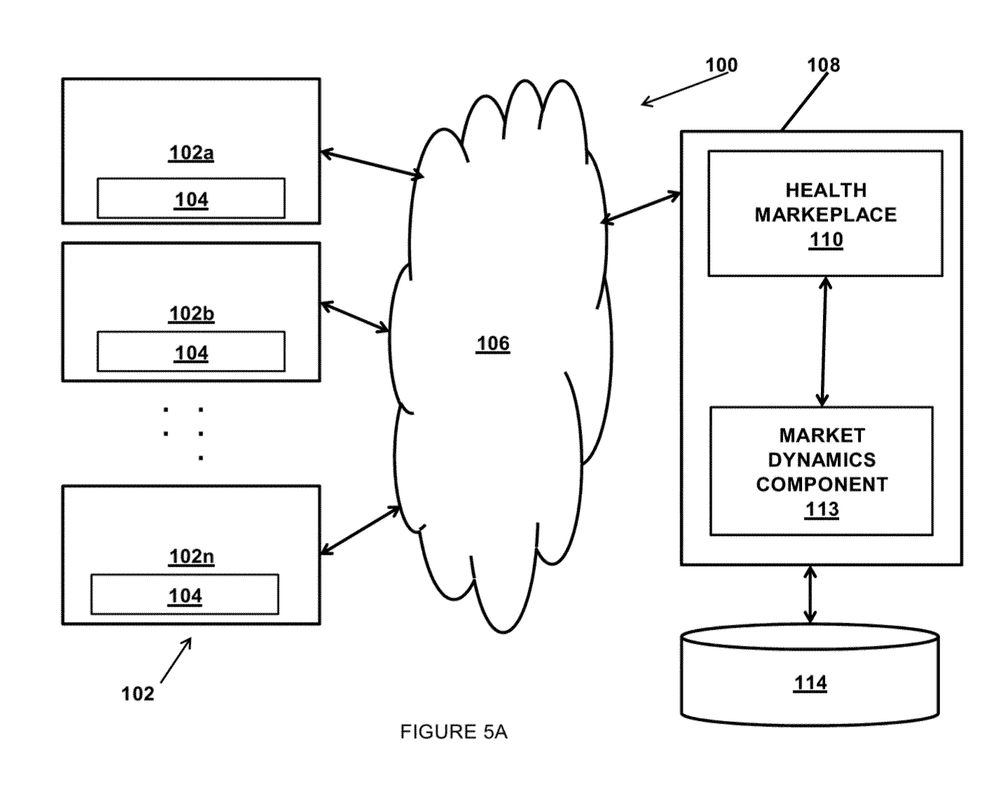

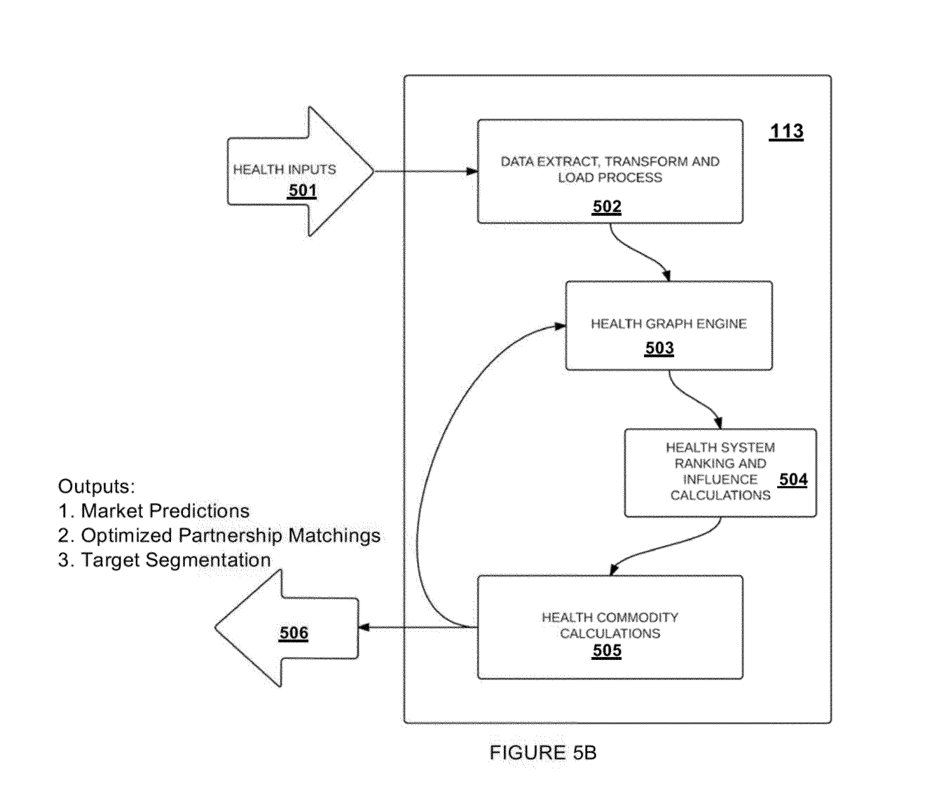

FIG. 5A illustrates an example of a health network system 100 that may incorporate a market dynamics component 113. In the example in FIG. 5A, the system may be implemented as a client/server, software as a service (SaaS) or a cloud based architecture. However, the system and in particular the market dynamics component 113 may also be implemented on a standalone computer that performs the operations of the market dynamics component 113 as described below or the market dynamics component 113 may be integrated into other systems.

In one implementation, as shown in FIG. 5A, the market dynamics component 113 may be integrated into a health system 100 in which one or more computing devices 102 may couple to, access and interface with a backend component 108 over a communications path 106. The backend component 108 may include a health marketplace 110 and the market dynamics component 113. The health marketplace may permit a user of the computing device to perform various health care related activities including shopping for health care, participating a health care blogs and forums and the like. The market dynamics component 113 may calculate the rate of change for the influence of supply and demand with respect to several multi-variate factors for the practitioner, payer and consumer alike in a health network. The detailed operation of the market dynamics component 113 is described below in more detail.

In the system, each computing device 102, such as computing devices 102a, 102b, . . . , 102n, may be a processor based device with memory, persistent storage, wired or wireless communication circuits and a display that allows each computing device to connect to and couple over the communication path 106 to a backend component 108. For example, each computing device may be a smartphone device, such as an Apple Computer product, Android OS based product, etc., a tablet computer, a personal computer, a terminal device, a laptop computer and the like. In one embodiment shown in FIG. 5A, each computing device 102 may store an application 104 in memory and then execute that application using the processor of the computing device to interface with the backend component 108. For example, the application may be a typical browser application or may be a mobile application. The communication path 106 may be a wired or wireless communication path that uses a secure protocol or an unsecure protocol. For example, the communication path 106 may be the Internet, Ethernet, a wireless data network, a cellular digital data network, a WiFi network and the like. The system 100 may also have a storage 114 that may be connected to the backend component 108 and may store various data, information and code that is part of the system.

The backend component 108 may be implemented using one or more computing resources, such as a processor, memory, flash memory, a hard disk drive, a blade server, an application server, a database server, a server computer, cloud computing resources and the like. The health marketplace 110 and the market dynamics component 113 may each be implemented in software or hardware. When the health marketplace 110 and the market dynamics component 113 are each implemented in software, each component may be a plurality of lines of computer code that reside on the backend component 108 (or are downloaded to the backend component 108) and may be executed by a processor of the backend component 108 so that the processor is configured to perform the operations of the health marketplace 110 or the market dynamics component 113. When the health marketplace 110 and the market dynamics component 113 and each implemented in hardware, each component may be an application specific integrated circuit, a microcontroller, a programmable logic device and the like that perform the operations of the health marketplace 110 or the market dynamics component 113.

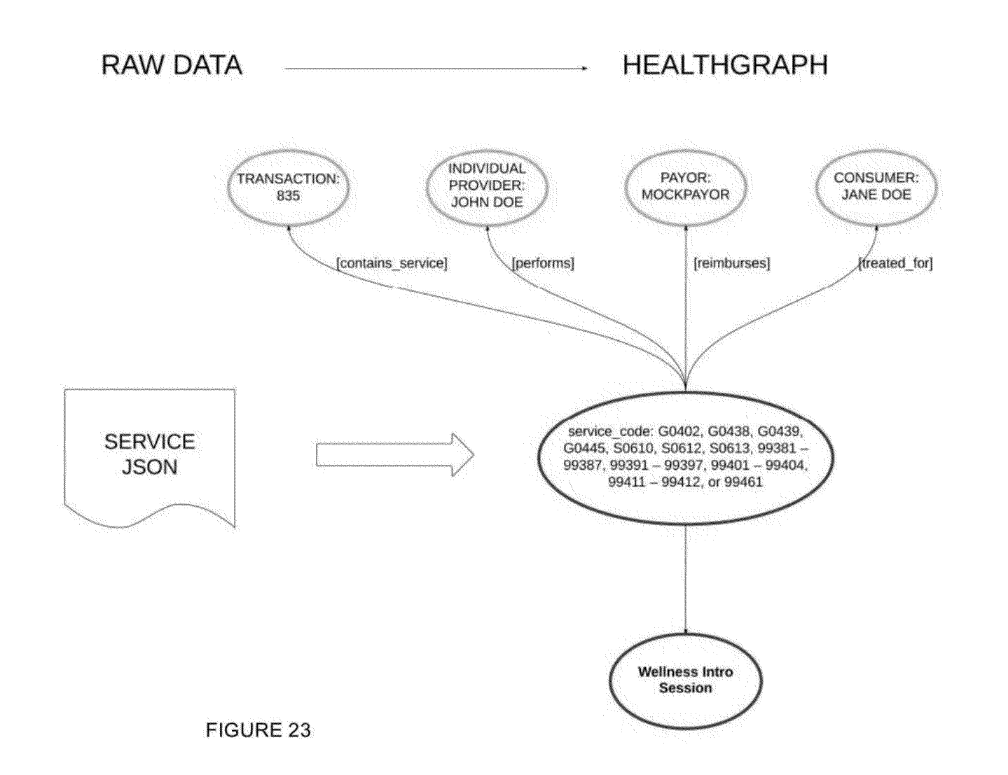

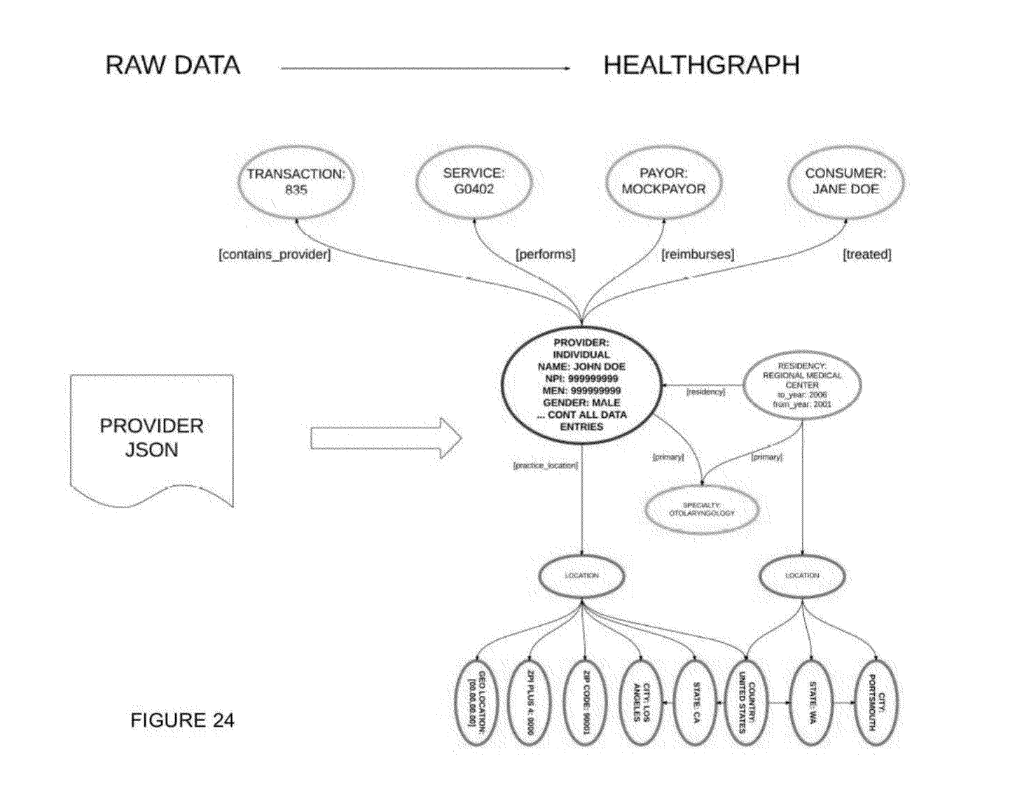

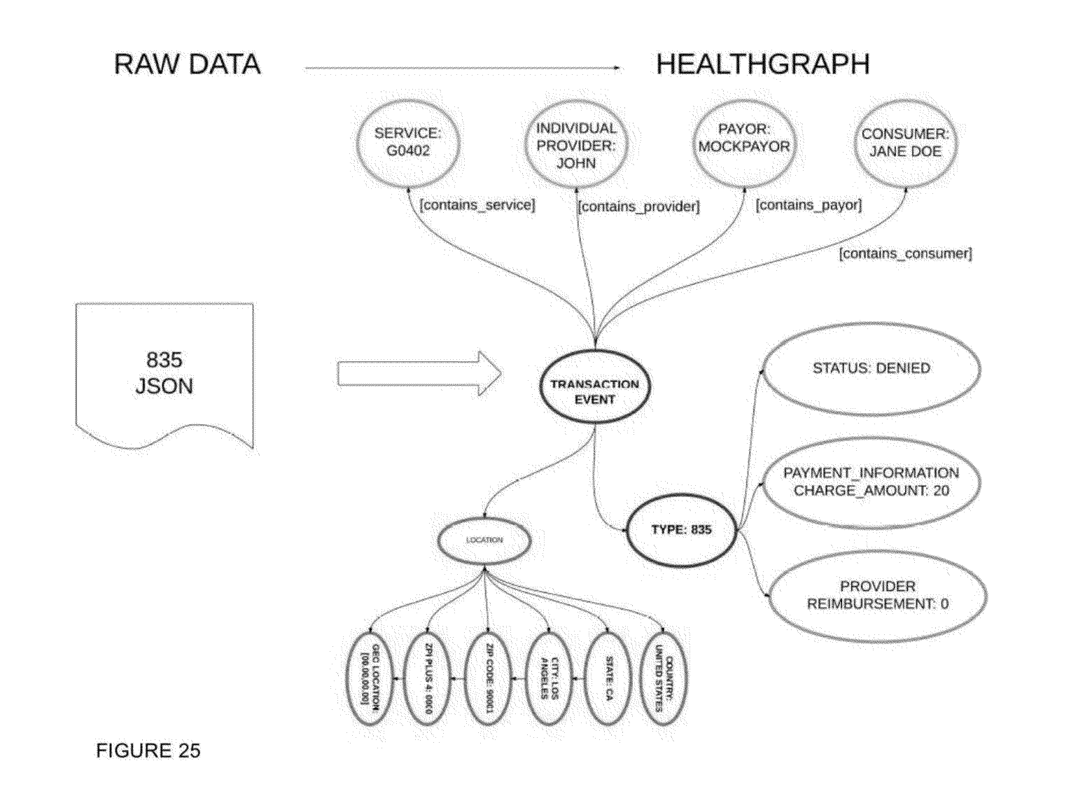

FIG. 5B illustrates more details of a market dynamic component 113. The component 113 may ingest internal and external data 501 as the observed health inputs. The health inputs 501 may be processed and normalized (using a data extract, transform and load process 502) into a suitable data format and the normalized health inputs may be fed into a health graph engine 503 (a graph database and analytics engine). The health graph engine 503 may then calculate entity rankings within each homogeneous system (using for example, Equation 2 described above in a health system ranking and influence calculation process 504.) Then, each entity's ranking is used to model commodity flow (using a health commodity calculations process 505) between the heterogeneous system connections described in FIG. 4. The commodity flow calculations are dynamically used to update the system's rankings via a database update in the system diagram. Additionally, the commodity flow calculations may be applied to create outputs 506, such as market predictions, partnership matchings, targeted market segmentation and other related recommendations. Appendix B, that is incorporated herein by reference, contains three raw health inputs to the system. Furthermore, FIGS. 23-25 described below, illustrate the process carried out by the apparatus shown in FIG. 5B. In addition, FIG. 26 described below applies these processes to demonstrate the exchange of commodities.

An example of the specific update and feedback loops of the market dynamics component are detailed in FIG. 6. As shown, the data sources 501 (shown as health inputs) may include internal observations, external rankings and healthcare transactions. The data source 501 may be processed for homogeneous rankings from within each entity's silo (as perform by process 504 described above). As shown in the health care example, each homogeneous system may be a payor system (and dynamic payor influence scoring generated for that homogeneous system), a provider system (and dynamic provider influence scoring generated for that homogeneous system), a consumer system (and dynamic consumer influence scoring generated for that homogeneous system), a transaction system (and dynamic transaction influence scoring generated for that homogeneous system) and service system (and dynamic service influence scoring generated for that homogeneous system).

As shown , these rankings are combined to assess commodity predictions (commodity assessment process 504). In turn, these recommendations are simultaneously used to update the internal ranking system while driving external recommendations 506. The actions taken, or not taken, for each recommendation create additional data points for further refinement of this system. For example, recommendation scores are shown in FIG. 8 [ A scored 1, B scored 2, C scored 1] In addition, FIG. 8A now applies the demand update rule via decaying eligibility traces according to (1) recommendations calculated from the ranking system of FIG. 8 and then (2) the classification of recommendations as action or no-action. All is framed within a provider referral system, as an example.

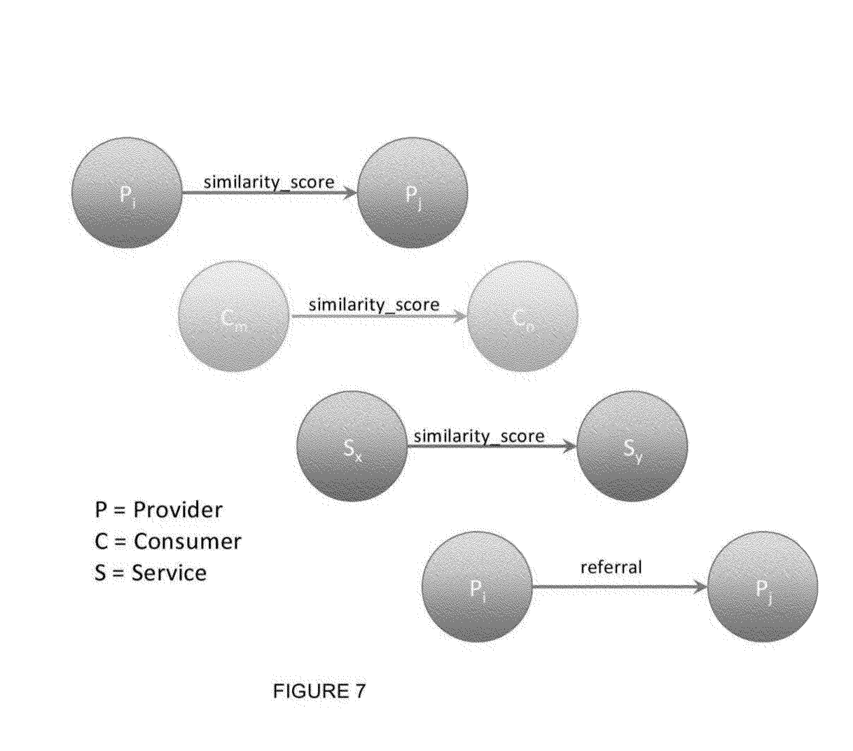

FIG. 7 demonstrates the internal calculation and storage of the similarity across entities within the same homogeneous system. Thus, as shown in FIG. 7, there may be a similarity score between two providers (who are both part of the provider network system), a similarity score between consumers (who are both part of the consumer network system), a similarity score between services (who are both part of the services network system) and a referral between two providers.

Based on common properties within each system, the system may apply various techniques in similarity scoring and entity ranking to dynamically calculate each vertex's total influence from within its network. For example, FIG. 8 demonstrates a methodology for using the property graph structure of the homogeneous provider sub-network to rank a set of entities. A similar process may be used for ranking the other entities of the health care system. In this example, the system may rank a set of providers according to the shared properties amongst them; specifically, the system and method may use graph traversals to rank providers according to shared specialties. FIG. 8 demonstrates one step of this traversal. As shown in FIG. 8, each provider may be initialized (as shown by the circles with empty centers) and may be assigned a rank as shown by the filled in circles based on the specialties using the pseudocode that appears in FIG. 8.

The method for determining the entity rank may be looped over the traversal to estimate the eigenvectors associated with the pagerank of this same system (as example of which is described in White, Scott, and Padhraic Smyth. “Algorithms for estimating relative importance in networks.” Proceedings of the ninth ACM SIGKDD international conference on Knowledge discovery and data mining. ACM, 2003 which is incorporated herein by reference).

The system may then apply the internal rankings across homogeneous systems (an example of which is shown in FIG. 8) as inputs for measuring the likelihood of interactions across entities in the larger healthcare network graph (process 504 in FIG. 5B described above). For example, FIG. 7 demonstrates a few of the internal interactions that the system can collect about each of the entities in the health system. The system can then apply similarity measures, like those outlined in FIG. 8, to identify and recommend similar heterogeneous connections. For example, the system may collect service and transaction information for provider Pₖ of FIG. 7. Over time, the system and method may aggregate this information to calculate the likelihood of a similar transaction occurring between similar endpoints in the network.

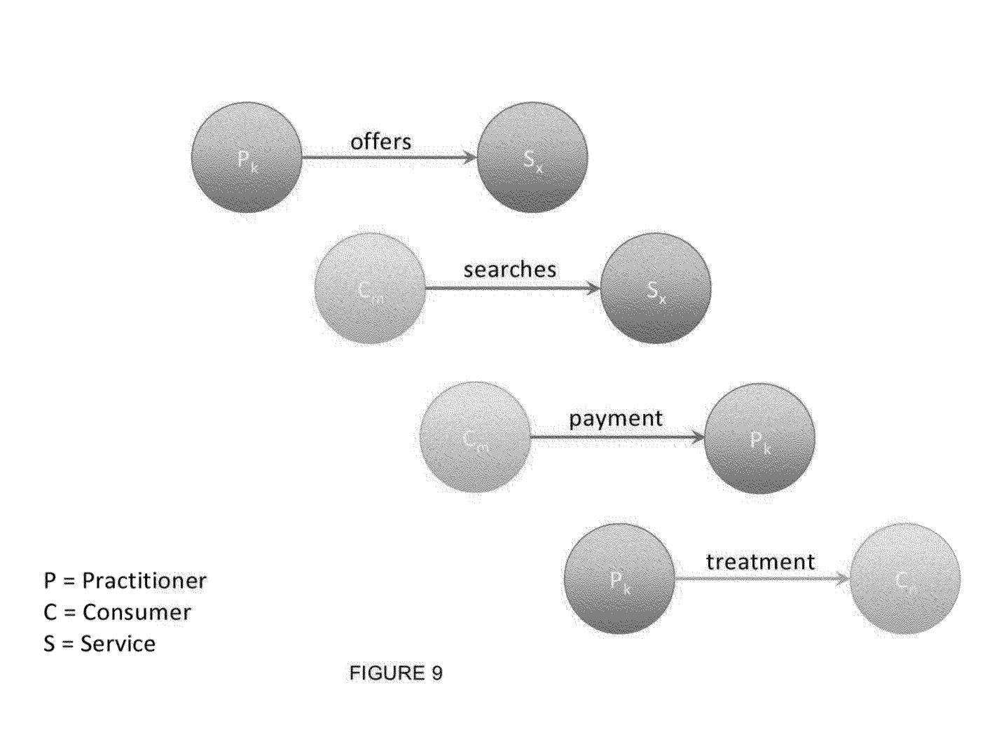

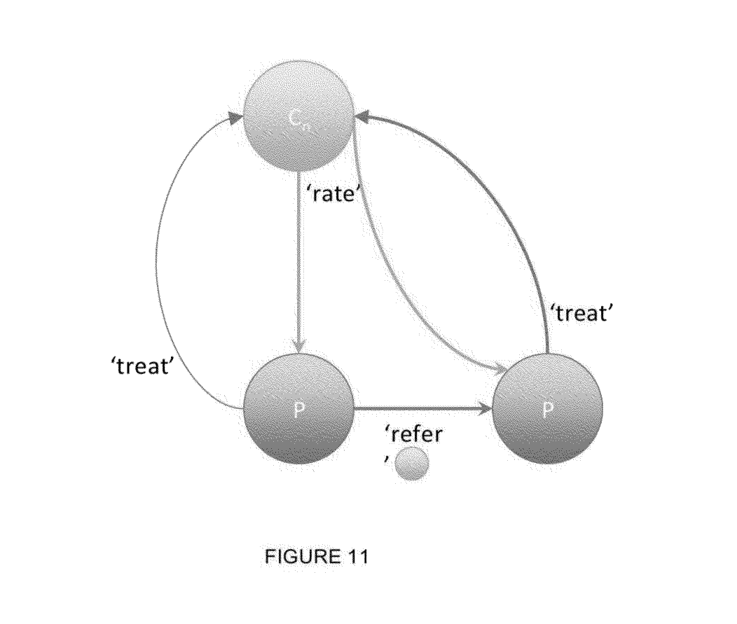

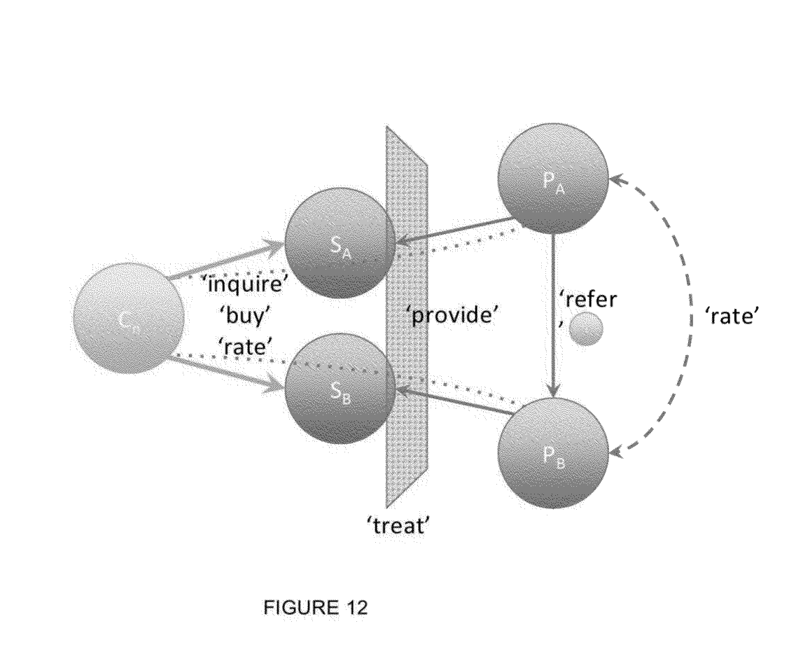

FIG. 9 illustrates an example of a set of heterogeneous relationships in a health care network and FIGS. 11 and 12 illustrates examples of heterogeneous influence models that show an example of observable information exchange within the healthcare network graph. The relationships may include offers between practitioners and services, searches between consumers and services, payment between consumers and practitioners and treatment between the practitioner and the consumer as shown in FIG. 9. The system may use the same methodology as described above and apply homogeneous rankings to determine the influence each respective entity will have within the entire system. Then, the system collects healthcare transactions across the heterogeneous system to update the influence of each entity and the corresponding likelihood of the observed transaction. As demonstrated in FIG. 6, these interactions will feed back into the system to update each vertex's influence within the system so that the system and method can identify new market recommendations.

As shown in FIG. 11, a consumer Cn and Practitioners P have the information exchanges (‘treat”, ‘rate’ and ‘refer’) and relationships shown. For example, the consumer may rate each practitioner after a treatment, each practitioner may treat the consumer and the practitioners may refer the consumer to each other. As shown in FIG. 12, treatments are real-world edges, but consumers only ‘indirectly’ rate practitioners by rating the services (S_{A} and S_{B} in FIG. 12) they provide. Practitioners also rate each other, confidentially. Our system makes use of ratings data to scale price and ‘marketing density’ for each practitioner.

Returning to FIG. 5B, the system and method uses a vertex's influence (generated by the health graph engine 503) to estimate a solution to the multicommodity flow problem and create a ranking system for each homogenous set of vertices. This stacked ranking yields an ordering from most to least influential for each of the sets outlined in Equation 2 above. The system and method may then translate this ranking as the upper bound for influential capacity for each vertex. The system may then estimate multicommodity flow into the immediate neighborhood of a vertex's network by augmenting the total flow of an edge according to the proportional influential capacity of each vertex. Given the upper bound of influential capacity for a vertex, the system and method estimates the local flow of multiple commodities to yield an approximation to the Dynamic Multicommodity Network via local approximation of the Maximal Diffusion Algorithm.

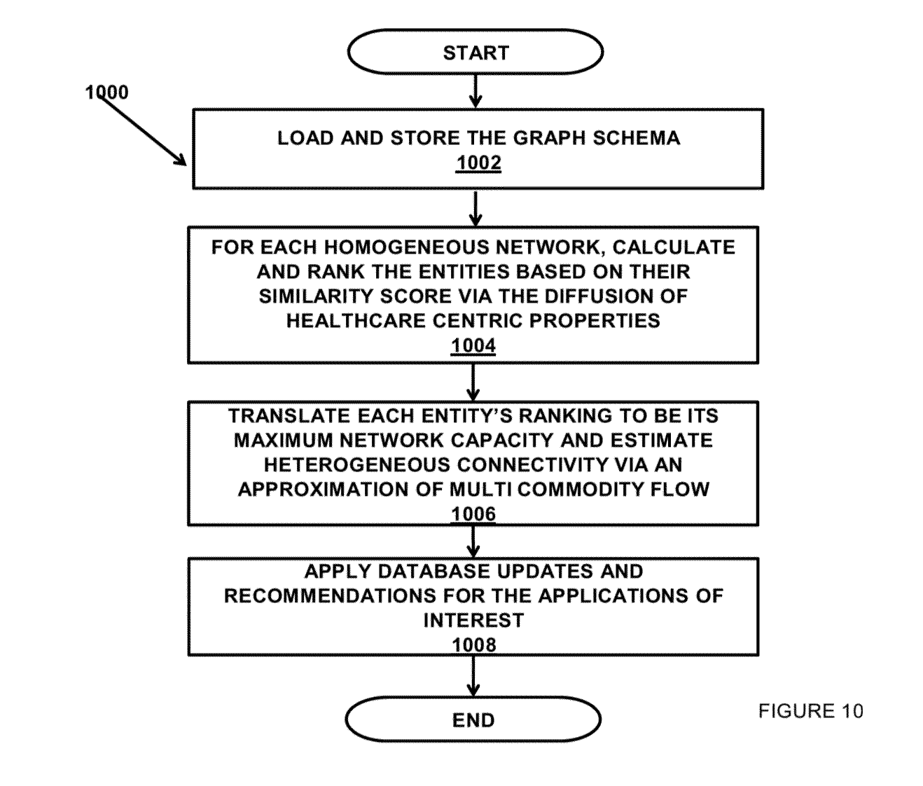

An example of the above process is now provided. The system and method for computing the multicommodity flow for the dynamic influence of a healthcare network contains the following the processes shown in FIG. 10. FIG. 10 illustrates a method 1000 for computing the multicommodity flow for the dynamic influence of a healthcare network. In one embodiment, the processes shown in FIG. 10 may be performed by the market dynamics component 113 in FIG. 5B and the sub-components of the market dynamics component 113. The method may load and store the graph schema (1002) such as by the health graph engine 502 shown in FIG. 5B. The method may, for each homogeneous network, calculate and rank the entities based on their similarity score via the diffusion of healthcare centric properties (1004) and this process may be performed, in one embodiment, by the health system ranking component 504 shown in FIG. 5B. The method may then translate each entity's ranking to be its maximum network capacity and estimate heterogeneous connectivity via an approximation of multi commodity flow (1006) and this process may be performed, in one embodiment, by the health commodity calculations component 505 shown in FIG. 5B. The method may then apply database updates and recommendations for the applications of interest (1008) such as the outputs 506 shown in FIG. 5B.

Graph Schema Construction and Process

Now, the graph schema construction and its process (process 1002 above that may be carried out by the health graph engine 503 in FIG. 5B) are described in more detail. The system may have/use five different schema models for the homogeneous systems described above.

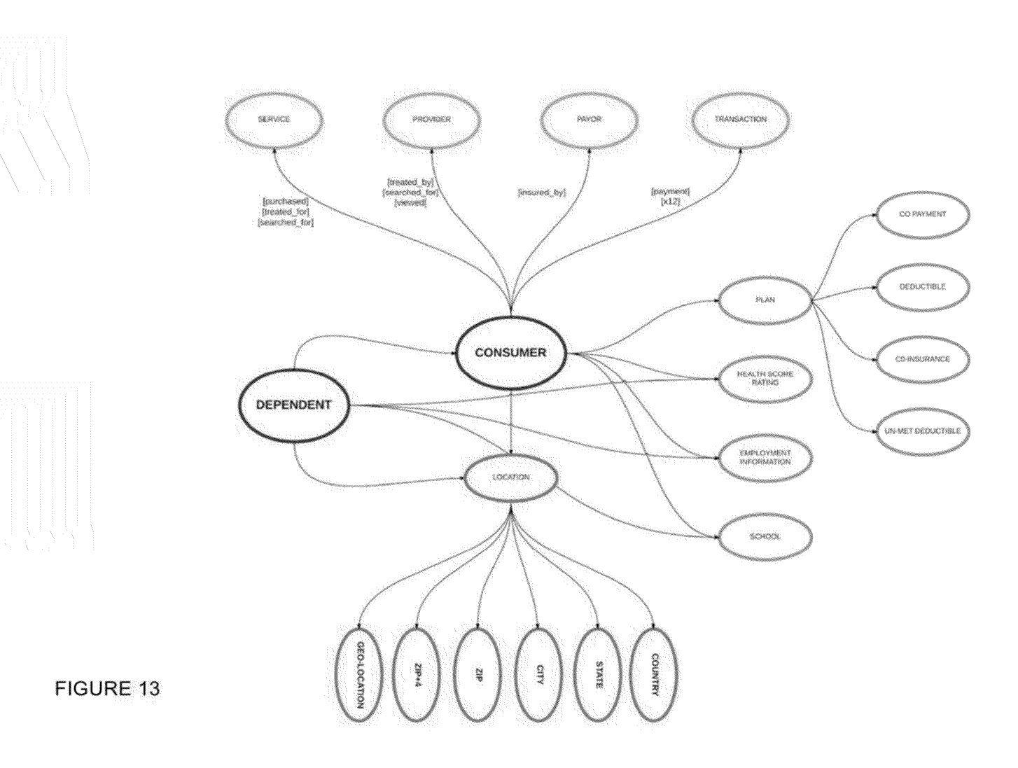

FIG. 13 illustrates an example of a schema for the graph construction and storage centered on the consumer model. Each addition to the consumer driver model is required to have the data listed in Table 10.1. The consumer model must have at least one element listed in Table 10.2. The optional properties and connections for the consumer driven model are listed in Table 10.3.

TABLE 10.1
Required objects for the consumer model

| | | Data Type or |
|---|---|---|
| Entity Name | Schema Type | Multiplicity |
| Consumer | Vertex | multiplicity: single |
| consumer_id | property | string |
| health_score | property | double |

TABLE 10.2
At least one of the following is a required
object for the consumer model

| | | Data Type or |
|---|---|---|
| Entity Name | Schema Type | Multiplicity |
| purchased | Edge | multiplicity: MULTI |
| consumer_id | Edge | multiplicity: MULTI |
| treated_for | Edge | multiplicity: MULTI |
| searched_for | Edge | multiplicity: MULTI |
| Viewed | Edge | multiplicity: MULTI |
| treated_by | Edge | multiplicity: MULTI |
| insured_by | Edge | multiplicity: MULTI |
| Payment | Edge | multiplicity: MULTI |
| [x12] | Edge | multiplicity: MULTI |

TABLE 10.3
Optional objects for the consumer model

| | | Data Type and/or |
|---|---|---|
| Entity Name | Schema Type | Multiplicity |
| Dependent | Vertex | multiplicity: multi |
| consumer_name | property | String |
| | | multiplicity: multi |
| dependent_name | property | String |
| | | multiplicity: multi |
| Location | vertex | multiplicity: multi |
| country_name | vertex | multiplicity: multi |
| state_name | vertex | multiplicity: multi |
| city_name | vertex | multiplicity: multi |
| zip_code | vertex | multiplicity: multi |
| zip_plus4_code | vertex | multiplicity: multi |
| geo_location | vertex | multiplicity: multi |
| Latitude | property | double |
| | | multiplicity: single |
| Longitude | property | double |
| | | multiplicity: single |
| Plan | vertex | multiplicity: multi |
| plan_id | property | string |
| | | multiplicity: multi |
| co_payment | vertex | multiplicity: multi |
| co_payment_amount | property | double |
| | | multiplicity: single |
| Deductible | vertex | multiplicity: multi |
| deductible_amount | property | double |
| | | multiplicity: single |
| co_insurance | vertex | multiplicity: multi |
| co_insurance_amount | property | double |
| | | multiplicity: single |
| unmet_deductible | vertex | multiplicity: multi |
| unmet_deductible_amount | property | double |
| | | multiplicity: single |
| employment_information | vertex | multiplicity: multi |
| employer_name | property | string |
| | | multiplicity: single |
| school | vertex | multiplicity: multi |
| school_name | property | string |
| | | multiplicity: single |

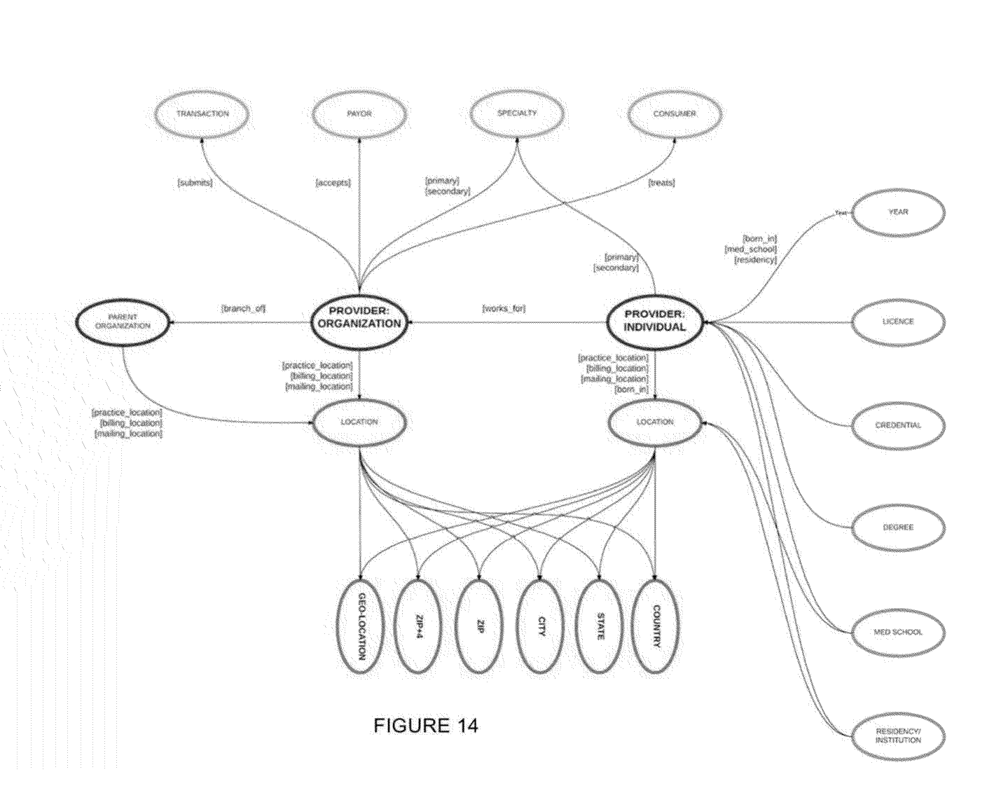

FIG. 14 illustrates an example of a schema for the graph construction and storage centered on the provider model. Each addition to the provider driver model is required to have the data listed in Table 11.1. The provider model must have at least one element listed in Table 11.2. The optional properties and connections for the provider driven model are listed in Table 11.3.

TABLE 11.1
Required objects for the provider model

| | | Data Type or |
|---|---|---|
| Entity Name | Schema Type | Multiplicity |
| provider | vertex | multiplicity: single |
| provider_organization | vertex | multiplicity: single |
| provider_id | property | string |
| | | multiplicity: single |

TABLE 11.2
At least one of the following is a required
objects for the provider model

| | | Data Type or |
|---|---|---|
| Entity Name | Schema Type | Multiplicity |
| Submitted | Edge | multiplicity: MULTI |
| Accepts | Edge | multiplicity: MULTI |
| Primary | Edge | multiplicity: MULTI |
| Secondary | Edge | multiplicity: MULTI |
| Treated | Edge | multiplicity: MULTI |

TABLE 11.3
Optional objects for the provider model

| | | Data Type and/or |
|---|---|---|
| Entity Name | Schema Type | Multiplicity |
| parent_organization | vertex | multiplicity: multi |
| parent_organization_name | property | string |
| | | multiplicity: single |
| located_in | edge | multiplicity: multi |
| Location | vertex | multiplicity: multi |
| country_name | vertex | multiplicity: multi |
| state_name | vertex | multiplicity: multi |
| city_name | vertex | multiplicity: multi |
| zip_code | vertex | multiplicity: multi |
| zip_plus4_code | vertex | multiplicity: multi |
| geo_location | vertex | multiplicity: multi |
| latitude | property | double |
| | | multiplicity: single |
| longitude | property | double |
| | | multiplicity: single |
| practice_location | edge | multiplicity: multi |
| billing_location | edge | multiplicity: multi |
| mailing_location | edge | multiplicity: multi |
| npi | property | multiplicity: single |
| men | property | multiplicity: single |
| year | vertex | multiplicity: multiple |
| year_value | property | integer |
| | | multiplicity: single |
| born_in | edge | multiplicity: single |
| graduated_in | edge | multiplicity: multi |
| license | vertex | multiplicity: multi |
| license_name | property | string |
| | | multiplicity: multi |
| license_practice | vertex | multiplicity: multi |
| license_practice_type | property | string |
| | | multiplicity: single |
| credential | vertex | multiplicity: multi |
| credential_type | property | string |
| | | multiplicity: single |
| degree | vertex | string |
| | | multiplicity: multi |
| degree_type | property | string |
| | | multiplicity: single |
| med_school | vertex | string |
| | | multiplicity: multi |
| med_school_name | property | string |
| | | multiplicity: single |
| residency | vertex | string |
| | | multiplicity: multi |
| residency_name | property | string |
| | | multiplicity: single |

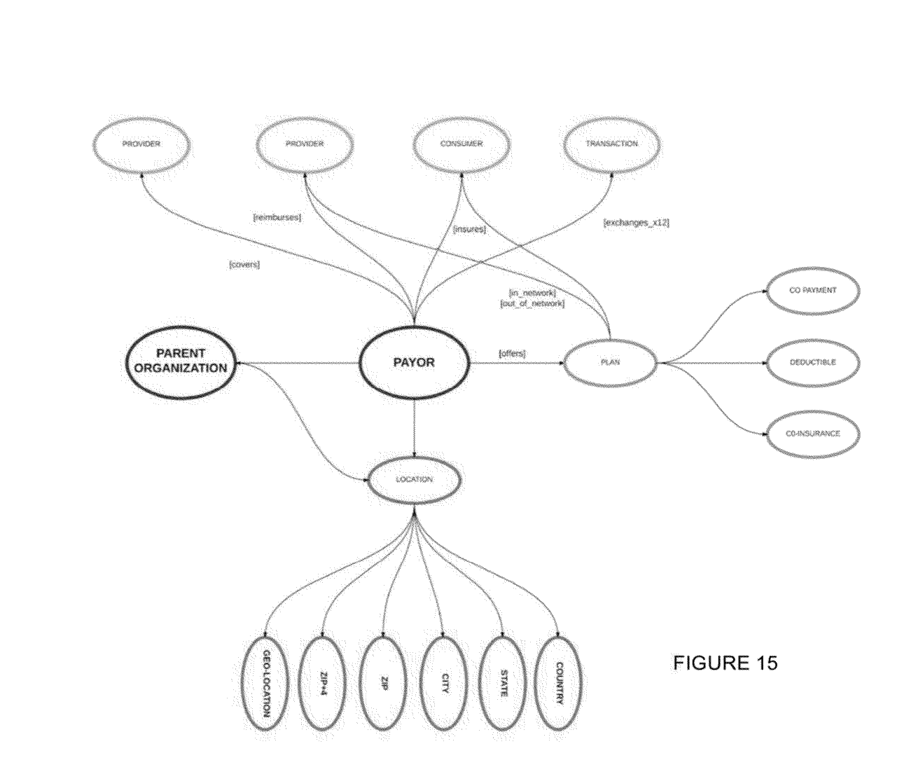

FIG. 15 illustrates an example of a schema for the graph construction and storage centered on the payor model. Each addition to the payor driver model is required to have the data listed in Table 12.1. The payor model must have at least one element listed in Table 12.2. The optional properties and connections for the payor driven model are listed in Table 12.3.

TABLE 12.1
Required objects for the payor model

| | | Data Type or |
|---|---|---|
| Entity Name | Schema Type | Multiplicity |
| payor | vertex | multiplicity: single |
| payor_id | property | string |
| | | multiplicity: single |
| offers | edge | multiplicity: multi |
| plan | vertex | multiplicity: multi |
| plan_id | property | string |
| | | multiplicity: single |

TABLE 12.2
At least one of the following is a required
objects for the payor model

| | | Data Type or |
|---|---|---|
| Entity Name | Schema Type | Multiplicity |
| Covers | Edge | multiplicity: MULTI |
| reimburses | Edge | multiplicity: MULTI |
| Insures | Edge | multiplicity: MULTI |
| in_network | Edge | multiplicity: MULTI |
| out_of_network | Edge | multiplicity: MULTI |
| exchanges_x12 | Edge | multiplicity: MULTI |

TABLE 12.3
Optional objects for the payor model

| | | Data Type and/or |
|---|---|---|
| Entity Name | Schema Type | Multiplicity |
| parent_organization | vertex | multiplicity: multi |
| parent_organization_name | property | string |
| | | multiplicity: single |
| located_in | edge | multiplicity: multi |
| location | vertex | multiplicity: multi |
| country_name | vertex | multiplicity: multi |
| state_name | vertex | multiplicity: multi |
| city_name | vertex | multiplicity: multi |
| zip_code | vertex | multiplicity: multi |
| zip_plus4_code | vertex | multiplicity: multi |
| geo_location | vertex | multiplicity: multi |
| latitude | property | double |
| | | multiplicity: single |
| longitude | property | double |
| | | multiplicity: single |
| co_payment | vertex | multiplicity: multi |
| co_payment_amount | property | double |
| | | multiplicity: single |
| deductible | vertex | multiplicity: multi |
| deductible_amount | property | double |
| | | multiplicity: single |
| co_insurance | vertex | multiplicity: multi |
| co_insurance_amount | property | double |
| | | multiplicity: single |

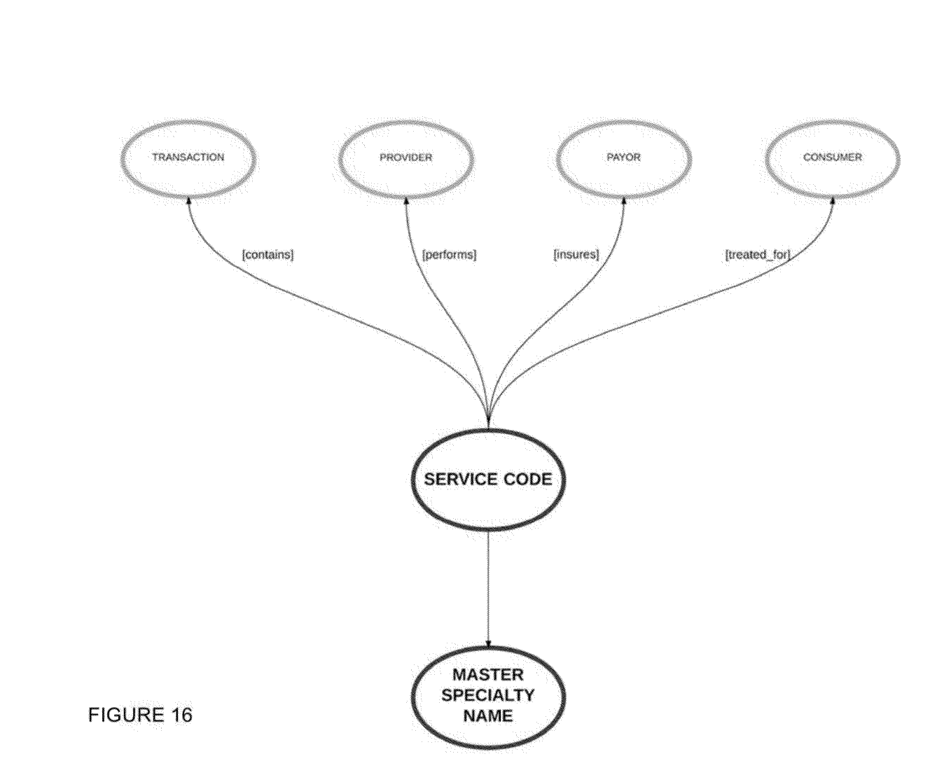

FIG. 16 illustrates an example of a schema for the graph construction and storage centered on the service model. Each addition to the service driver model is required to have the data listed in Table 13.1. The service model must have at least one element listed in Table 13.2.

TABLE 13.1
Required objects for the service model

| | | Data Type or |
|---|---|---|
| Entity Name | Schema Type | Multiplicity |
| service | vertex | multiplicity: multi |
| service_code | property | string |
| | | multiplicity: single |
| master_specialty | vertex | multiplicity: multi |
| master_specialty_name | property | string |
| | | multiplicity: single |

TABLE 13.2
At least one of the following is a required
objects for the service model

| | | Data Type or |
|---|---|---|
| Entity Name | Schema Type | Multiplicity |
| contains | Edge | multiplicity: MULTI |
| performs | Edge | multiplicity: MULTI |
| insures | Edge | multiplicity: MULTI |
| treated_for | Edge | multiplicity: MULTI |

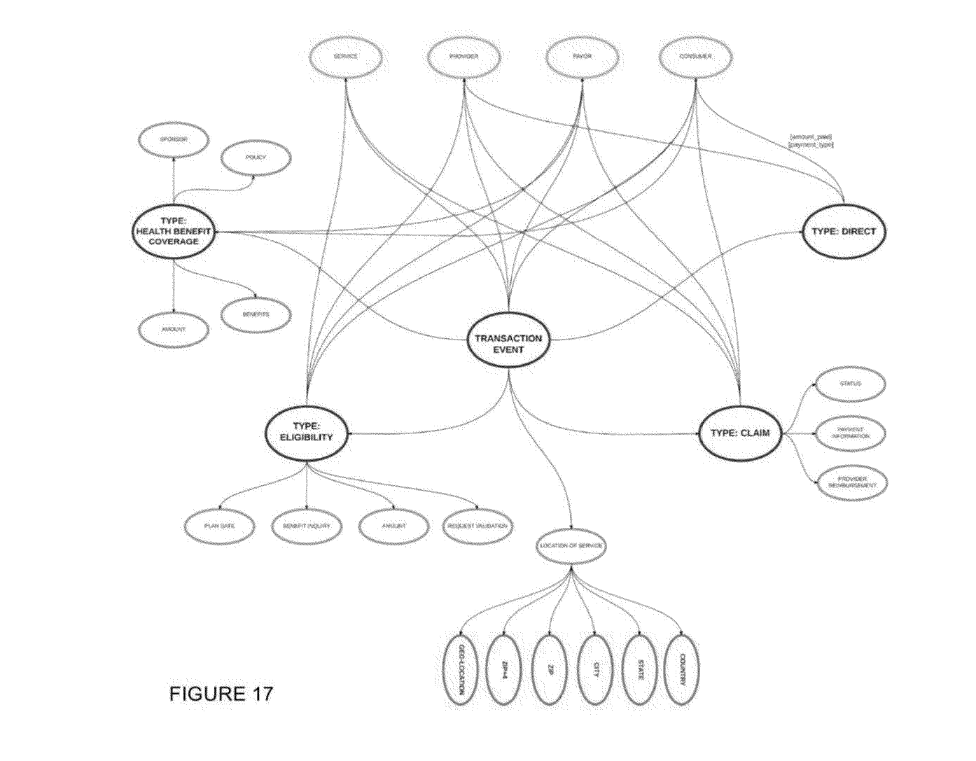

FIG. 17 illustrates an example of a schema for the graph construction and storage centered on the transaction model. Each addition to the transaction model is required to have the data listed in Table 14.1. The transaction model must have at least one element listed in Table 14.2. The optional properties and connections for the transaction model are listed in Table 14.3.

TABLE 14.1
Required objects for the transaction model

| | | Data Type or |
|---|---|---|
| Entity Name | Schema Type | Multiplicity |
| transaction_event | vertex | multiplicity: single |
| transaction_event_name | property | string |
| | | multiplicity: single |
| transaction_type | vertex | multiplicity: single |
| contains | edge | multiplicity: multi |
| of_type | edge | multiplicity: single |

TABLE 14.2
At least one of the following is a required
objects for the transaction model

| | | Data Type or |
|---|---|---|
| Entity Name | Schema Type | Multiplicity |
| transaction_type_direct | property | string |
| | | multiplicity: single |
| transaction_type_hbc | property | string |
| | | multiplicity: single |
| transaction_type_eligibility | property | string |
| | | multiplicity: single |
| transaction_type_claim | property | string |
| | | multiplicity: single |
| transaction_type_direct | property | string |
| | | multiplicity: single |

TABLE 14.3
Optional objects for the transaction model

| | | Data Type and/or |
|---|---|---|
| Entity Name | Schema Type | Multiplicity |
| located_in | edge | multiplicity: multi |
| location_of_service | vertex | multiplicity: multi |
| country_name | vertex | multiplicity: multi |
| state_name | vertex | multiplicity: multi |
| city_name | vertex | multiplicity: multi |
| zip_code | vertex | multiplicity: multi |
| zip_plus4_code | vertex | multiplicity: multi |
| geo_location | vertex | multiplicity: multi |
| latitude | property | double |
| | | multiplicity: single |
| longitude | property | double |
| | | multiplicity: single |
| sponsor | vertex | multiplicity: multi |
| sponsor_name | property | string |
| | | multiplicity: single |
| policy | vertex | multiplicity: multi |
| policy_id | property | string |
| | | multiplicity: single |
| benefit | vertex | multiplicity: multi |
| benefit_id | property | string |
| | | multiplicity: single |
| amount | vertex | multiplicity: multi |
| amount_value | property | double |
| | | multiplicity: single |
| plan | vertex | multiplicity: multi |
| plan_date | property | date time |
| | | multiplicity: single |
| benefit_inquiry | vertex | multiplicity: multi |
| benefit_inqury_id | property | string |
| | | multiplicity: single |
| request_validation | vertex | multiplicity: multi |
| request_validation_value | property | string |
| | | multiplicity: single |
| status | vertex | multiplicity: multi |
| status_id | property | string |
| | | multiplicity: single |
| payment | vertex | multiplicity: multi |
| payment_information | property | double |
| | | multiplicity: single |
| provider_reimbursement | vertex | multiplicity: multi |
| provider_reimbursement_value | property | double |
| | | multiplicity: single |

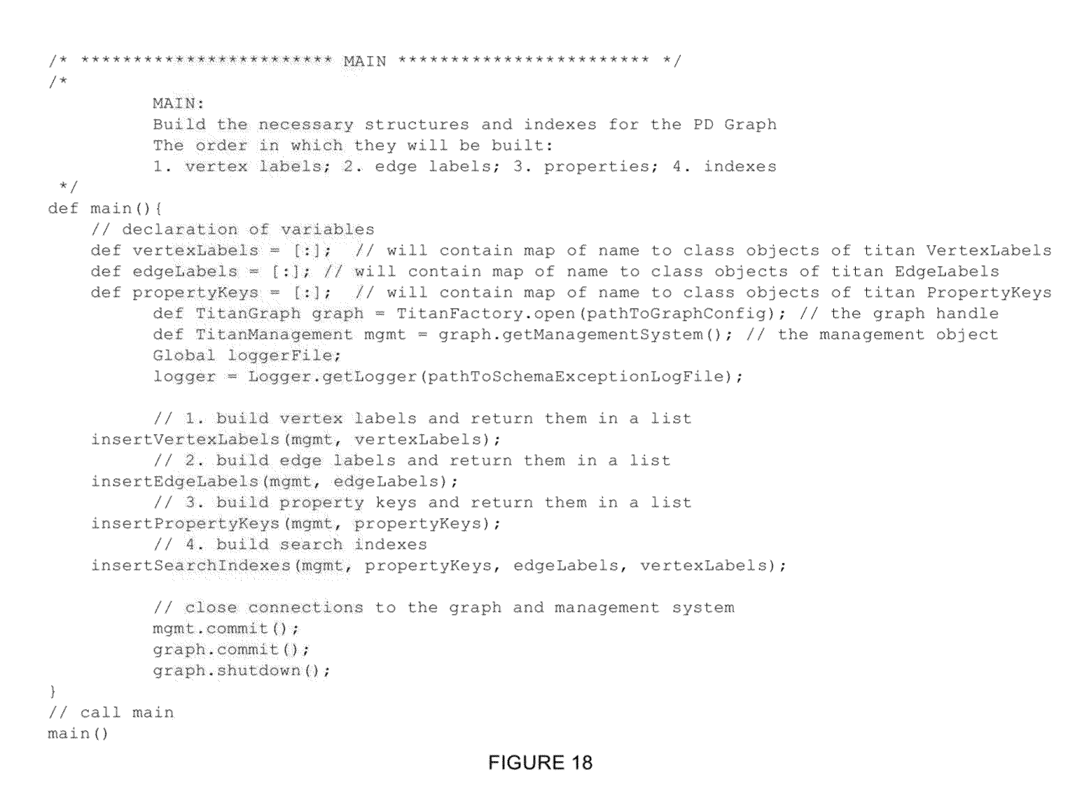

FIG. 18 is an example of the code that may create the schema and load the schema into a graph. The code details the schema creation and load into a graph database. The code is instantiated with the above schema to prepare and load the data.

Calculate Similarity Scores and Entity Ranks for Homogeneous System

An example of the code that may be used to calculate similarity scores and entity rank is included in Appendix A that is incorporated herein by reference. In particular, the code in Appendix A calculates the ranking and diffusion of influence for providers according to the structure of their homogeneous network via basic best mode algorithm. The influence scores can be calculated according to a single property, the Euclidean distance between primary practice locations, or a combination. The final similarity score is represented either as a vote or a continuous likelihood which ranges between the min and max observed similarity scores.

Calculate the Maximum Network Capacity for Heterogeneous Connectivity for Multiple Commodities

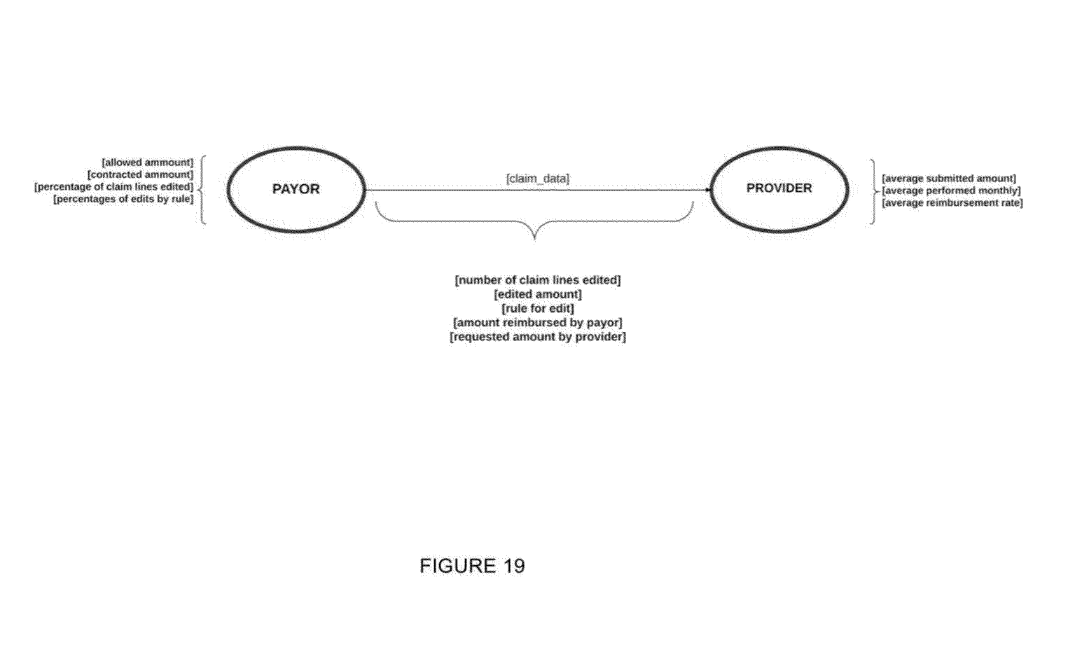

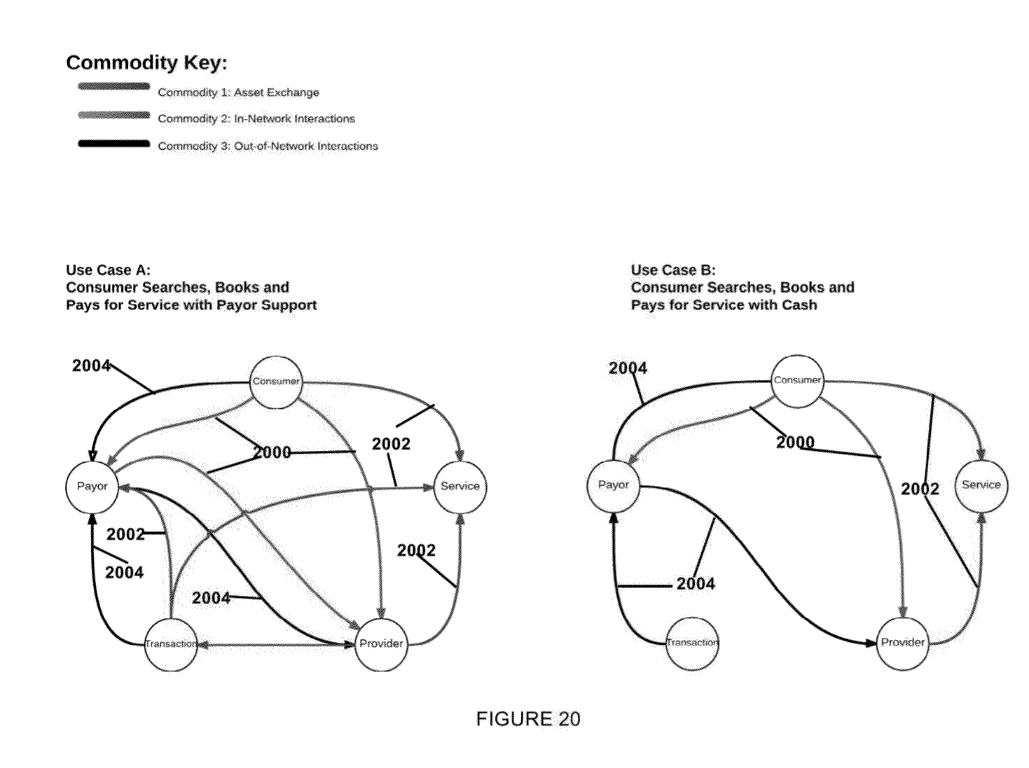

The system and method translate the influence ranking from within each vertex system to instantiate the estimation of multicommodity flow throughout the network. FIG. 19 illustrates an example of the data interchange between two entities in the healthcare network and shows data of the payor and data of the provider and then claim_data between the entities. FIG. 20 illustrates two different uses cases of a commodity network in the healthcare field. In particular, the commodities may be an asset exchange commodity 2000, an in-network interaction commodity 2002 and an out of network interaction commodity 2004. The example on FIG. 21 is an instance of a Dynamic Multicommodity Healthcare Network.

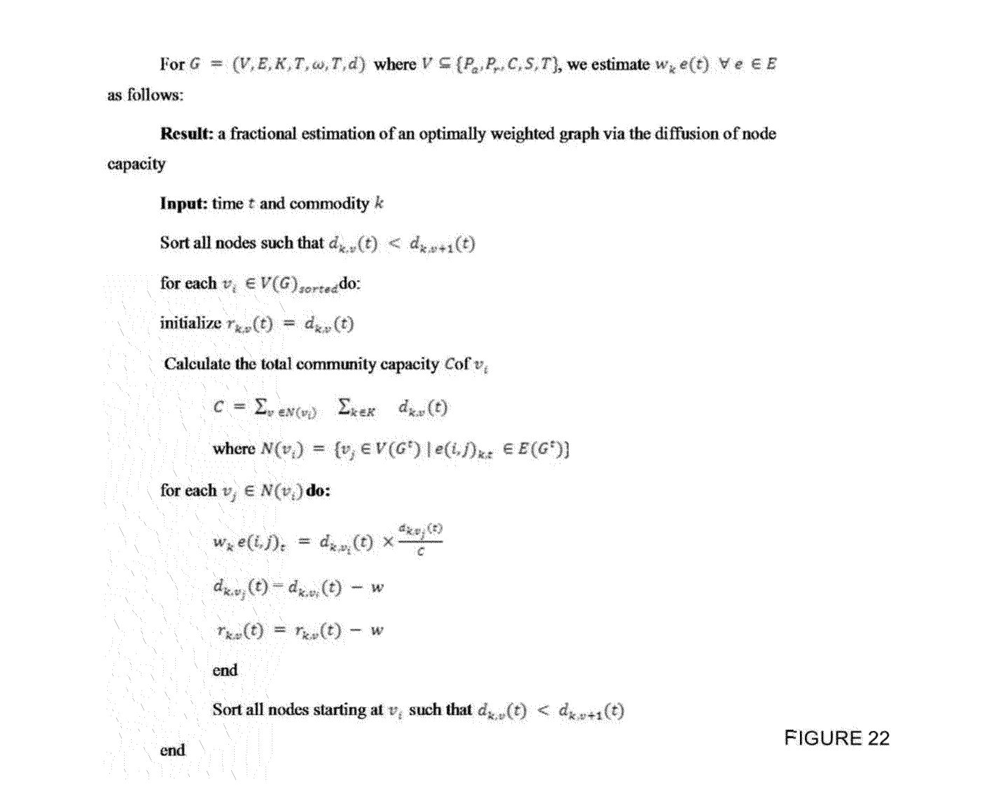

The system and method define ω_{k,∈}(t) to be the maximum edge capacity for flow of commodity k at time t. The system defines d_{k,i}(t) to be the total input capacity for vertex vᵢ, where d _{k,i}(t) is dynamically calculated to be the total influence vertex vᵢ has amongst its homogeneous system. The system and method seeks to assign each value wₖe(t) such that Σ_{v∈N(v)} rₖ(t)→0 where rₖ(t)=d_{k,N}(t)−Σ_{v∈N(v)} ω_{k,∈}(t). An example of the pseudocode for flow estimation used in the model is shown in FIG. 22.

Recommend, Segment, or Predict. Apply Update Rules for Dynamic Database Ranking and Capacity Estimation

Additional granularity augments this problem with the observation of transactional ratings and referrals, as demonstrated in FIG. 11. Herein, the system and method may apply an update rule:

dₖ_{ᵢ}ᵥ_{ᵢ}(tᵢ)←α f(tᵢ₋₁,kᵥ,dₜ₋₁,rₜ₋₁)

where α is a learned weight of the system's behavior, t represents time, k is specific to the commodity of interest, d is the total vertex demand at time t, and r is the rating of the transaction. FIGS. 5B and 6 depict the location of the update function with respect to the flow of the entire system.

The system and method may apply methodologies from reinforcement learning to learn the weight α as t→τ. The system may start by initializing alpha to be α=0.5 and apply eligibility traces to learn the dynamic weight of the update rule. The system and method may implement eligibility traces due to their useful nature for representing a short-term memory process which gradually decays over time. These traces give the system and method the ability to award a good (or bad) event by augmenting the total credit accordingly. Most generally, an accumulating trace is a trace which builds up over time when each state is visited with a decay parameter. The conventional definition for this update rule is as follows:

$${a}_{t + 1} ( s ) = \{ \frac{γ λ {α}_{t} ( s ) if s 1 = {s}_{t}}{1 if s = {s}_{t}}$$

where λ≦0≦1 is the value of decay and γ≦0≦1 is the discount-rate. The system and method can set γ and λ both to 1 to treat the system as a true accumulative trace with infinite memory, or the system and method may set γ and λ both to 0 to treat the update system as a Markovian model.

Result: An update procedure for tracking the dynamic influence score

Input: A recommendation as an edge e, decay parameter λ and the discount rate λ

For v₁ and v₂ ∈ e,
For each k ∈ K
dₖ_{_{i,}}ᵥ_{ᵢ}(t₀)←α=0.5

Observe trial at time tᵢ

Classify outcome of trial

For v₁ and v₂ ∈ e,
For each k ∈ K

$${a}_{t + 1} ( s ) = \{ \frac{γ λ {α}_{t} ( s ) if s 1 = {s}_{t}}{1 if s = {s}_{t}}$$

The foregoing description, for purpose of explanation, has been described with reference to specific embodiments. However, the illustrative discussions above are not intended to be exhaustive or to limit the disclosure to the precise forms disclosed. Many modifications and variations are possible in view of the above teachings. The embodiments were chosen and described in order to best explain the principles of the disclosure and its practical applications, to thereby enable others skilled in the art to best utilize the disclosure and various embodiments with various modifications as are suited to the particular use contemplated.

The system and method disclosed herein may be implemented via one or more components, systems, servers, appliances, other subcomponents, or distributed between such elements. When implemented as a system, such systems may include an/or involve, inter alia, components such as software modules, general-purpose CPU, RAM, etc. found in general-purpose computers,. In implementations where the innovations reside on a server, such a server may include or involve components such as CPU, RAM, etc., such as those found in general-purpose computers.

Additionally, the system and method herein may be achieved via implementations with disparate or entirely different software, hardware and/or firmware components, beyond that set forth above. With regard to such other components (e.g., software, processing components, etc.) and/or computer-readable media associated with or embodying the present inventions, for example, aspects of the innovations herein may be implemented consistent with numerous general purpose or special purpose computing systems or configurations. Various exemplary computing systems, environments, and/or configurations that may be suitable for use with the innovations herein may include, but are not limited to: software or other components within or embodied on personal computers, servers or server computing devices such as routing/connectivity components, hand-held or laptop devices, multiprocessor systems, microprocessor-based systems, set top boxes, consumer electronic devices, network PCs, other existing computer platforms, distributed computing environments that include one or more of the above systems or devices, etc.

In some instances, aspects of the system and method may be achieved via or performed by logic and/or logic instructions including program modules, executed in association with such components or circuitry, for example. In general, program modules may include routines, programs, objects, components, data structures, etc. that perform particular tasks or implement particular instructions herein. The inventions may also be practiced in the context of distributed software, computer, or circuit settings where circuitry is connected via communication buses, circuitry or links. In distributed settings, control/instructions may occur from both local and remote computer storage media including memory storage devices.

The software, circuitry and components herein may also include and/or utilize one or more type of computer readable media. Computer readable media can be any available media that is resident on, associable with, or can be accessed by such circuits and/or computing components. By way of example, and not limitation, computer readable media may comprise computer storage media and communication media. Computer storage media includes volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical storage, magnetic tape, magnetic disk storage or other magnetic storage devices, or any other medium which can be used to store the desired information and can accessed by computing component. Communication media may comprise computer readable instructions, data structures, program modules and/or other components. Further, communication media may include wired media such as a wired network or direct-wired connection, however no media of any such type herein includes transitory media. Combinations of the any of the above are also included within the scope of computer readable media.

In the present description, the terms component, module, device, etc. may refer to any type of logical or functional software elements, circuits, blocks and/or processes that may be implemented in a variety of ways. For example, the functions of various circuits and/or blocks can be combined with one another into any other number of modules. Each module may even be implemented as a software program stored on a tangible memory (e.g., random access memory, read only memory, CD-ROM memory, hard disk drive, etc.) to be read by a central processing unit to implement the functions of the innovations herein. Or, the modules can comprise programming instructions transmitted to a general purpose computer or to processing/graphics hardware via a transmission carrier wave. Also, the modules can be implemented as hardware logic circuitry implementing the functions encompassed by the innovations herein. Finally, the modules can be implemented using special purpose instructions (SIMD instructions), field programmable logic arrays or any mix thereof which provides the desired level performance and cost.

As disclosed herein, features consistent with the disclosure may be implemented via computer-hardware, software and/or firmware. For example, the systems and methods disclosed herein may be embodied in various forms including, for example, a data processor, such as a computer that also includes a database, digital electronic circuitry, firmware, software, or in combinations of them. Further, while some of the disclosed implementations describe specific hardware components, systems and methods consistent with the innovations herein may be implemented with any combination of hardware, software and/or firmware. Moreover, the above-noted features and other aspects and principles of the innovations herein may be implemented in various environments. Such environments and related applications may be specially constructed for performing the various routines, processes and/or operations according to the invention or they may include a general-purpose computer or computing platform selectively activated or reconfigured by code to provide the necessary functionality. The processes disclosed herein are not inherently related to any particular computer, network, architecture, environment, or other apparatus, and may be implemented by a suitable combination of hardware, software, and/or firmware. For example, various general-purpose machines may be used with programs written in accordance with teachings of the invention, or it may be more convenient to construct a specialized apparatus or system to perform the required methods and techniques.

Aspects of the method and system described herein, such as the logic, may also be implemented as functionality programmed into any of a variety of circuitry, including programmable logic devices (“PLDs”), such as field programmable gate arrays (“FPGAs”), programmable array logic (“PAL”) devices, electrically programmable logic and memory devices and standard cell-based devices, as well as application specific integrated circuits. Some other possibilities for implementing aspects include: memory devices, microcontrollers with memory (such as EEPROM), embedded microprocessors, firmware, software, etc. Furthermore, aspects may be embodied in microprocessors having software-based circuit emulation, discrete logic (sequential and combinatorial), custom devices, fuzzy (neural) logic, quantum devices, and hybrids of any of the above device types. The underlying device technologies may be provided in a variety of component types, e.g., metal-oxide semiconductor field-effect transistor (“MOSFET”) technologies like complementary metal-oxide semiconductor (“CMOS”), bipolar technologies like emitter-coupled logic (“ECL”), polymer technologies (e.g., silicon-conjugated polymer and metal-conjugated polymer-metal structures), mixed analog and digital, and so on.

It should also be noted that the various logic and/or functions disclosed herein may be enabled using any number of combinations of hardware, firmware, and/or as data and/or instructions embodied in various machine-readable or computer-readable media, in terms of their behavioral, register transfer, logic component, and/or other characteristics. Computer-readable media in which such formatted data and/or instructions may be embodied include, but are not limited to, non-volatile storage media in various forms (e.g., optical, magnetic or semiconductor storage media) though again does not include transitory media. Unless the context clearly requires otherwise, throughout the description, the words “comprise,” “comprising,” and the like are to be construed in an inclusive sense as opposed to an exclusive or exhaustive sense; that is to say, in a sense of “including, but not limited to.” Words using the singular or plural number also include the plural or singular number respectively. Additionally, the words “herein,” “hereunder,” “above,” “below,” and words of similar import refer to this application as a whole and not to any particular portions of this application. When the word “or” is used in reference to a list of two or more items, that word covers all of the following interpretations of the word: any of the items in the list, all of the items in the list and any combination of the items in the list.

Although certain presently preferred implementations of the invention have been specifically described herein, it will be apparent to those skilled in the art to which the invention pertains that variations and modifications of the various implementations shown and described herein may be made without departing from the spirit and scope of the invention. Accordingly, it is intended that the invention be limited only to the extent required by the applicable rules of law.

While the foregoing has been with reference to a particular embodiment of the disclosure, it will be appreciated by those skilled in the art that changes in this embodiment may be made without departing from the principles and spirit of the disclosure, the scope of which is defined by the appended claims.

1. An apparatus, comprising:
a processor and a memory;
the processor being configured to receive a plurality of health inputs, the plurality of health inputs associated with a plurality of health entities;
the processor being configured to calculate for each entity, an entity ranking within each homogeneous system using the plurality of health inputs associated with the plurality of health entities; and
the processor being configured to model a commodity flow for each health entity based on the ranking for each health entity.

2. The apparatus of claim 1 further comprising the processor being configured to generate one or more outputs based on the modeled commodity flow, the outputs being one of a market prediction, a partnership matching, a targeted market segmentation and a recommendation.

3. The apparatus of claim 1, wherein the processor being configured to calculate the entity ranking for each entity calculates the entity ranking for each entity using V ⊂ {P_{α},Pᵣ,C,S,T} where P_{A} represents one or more payors, Pᵣ represented one or more providers, C represents one or more consumers, S represents one or more services and T represents one or more transactions observed within the healthcare network.

4. The apparatus of claim 1, wherein each health input is a graph schema for each homogeneous system.

5. The apparatus of claim 4, wherein each homogeneous system is one of a provider network, a consumer network, a service network and a transaction network.

6. The apparatus of claim 1, wherein the processor being configured to calculate the entity ranks further comprises the processor being configured to diffuse the health inputs to generate the entity ranks.

7. The apparatus of claim 1, wherein the processor being configured to model a commodity flow further comprises the processor being configured to determine a maximum network capacity for each health entity.

8. The apparatus of claim 7, wherein the processor being configured to model a commodity flow further comprises the processor being configured to estimate a hetoergeneous system connectivity for each health entity.

9. A method, comprising:
receiving a plurality of health inputs, the plurality of health inputs associated with a plurality of health entities;
calculating, for each entity, an entity ranking within each homogeneous system using the plurality of health inputs associated with the plurality of health entities; and
modeling a commodity flow for each health entity based on the ranking for each health entity.

10. The method of claim 9 further comprising generating one or more outputs based on the modeled commodity flow, the outputs being one of a market predictions, a partnership matching, a targeted market segmentation and a recommendation.

11. The method of claim 9, wherein calculating the entity ranking for each entity further comprises calculating the entity ranking for each entity using V ⊂ {P_{α},Pᵣ,C,S,T} where P_{A} represents one or more payors, Pᵣ represented one or more providers, C represents one or more consumers, S represents one or more services and T represents one or more transactions observed within the healthcare network.

12. The method of claim 9, wherein each health input is a graph schema for each homogeneous system.

13. The method of claim 12, wherein each homogeneous system is one of a provider network, a consumer network, a service network and a transaction network.

14. The method of claim 9, wherein calculating the entity ranks further comprises diffusing the health inputs to generate the entity ranks.

15. The method of claim 9, wherein modeling the commodity flow further comprises determining a maximum network capacity for each health entity.

16. The method of claim 15, wherein modeling the commodity flow further comprises estimating a hetoergeneous system connectivity for each health entity.
